(19) United States
(12) Patent Application Publication
(10) Pub. No.: US 2014/0228278 A1
(43) Pub. Date: Aug. 14, 2014

(54) ANTIBIOTICS AND METHODS FOR MANUFACTURING THE SAME

(71) Applicant: THE BOARD OF TRUSTEES OF THE UNIVERSITY OF ILLINOIS, Urbana, IL (US)

(72) Inventors: Douglas A. Mitchell, Urbana, IL (US); Katie J. Molohon, Champaign, IL (US)

(21) Appl. No.: 14/129,837

(22) PCT Filed: Oct. 21, 2011
(86) PCT No.: PCT/US2011/057333

§ 371 Date: Apr. 23, 2014

Related U.S. Application Data
(60) Provisional application No. 61/501,412, filed on Jun. 27, 2011.

(51) Int. Cl. C07K 5/062; C12Q 1/18

(52) U.S. Cl. CPC C07K 5/06034; C12Q 1/18
USPC 514/2.4; 548/204; 514/2.3; 435/32; 435/71.3

(57) ABSTRACT

The present invention generally relates to a novel highly discriminating antibiotic, plantazolicin, a plantazolicin-like compound and to pharmaceutical compositions comprising the same. Also provided are methods for producing and using plantazolicin. Due to its bactericidal activity against Bacillus anthracis, plantazolicin and plantazolicin-like compounds can be used in methods for treating and/or preventing anthrax infections.

### FIELD OF THE INVENTION

The present invention generally relates to a novel highly discriminating antibiotic, plantazolicin (PZN), which was isolated from Bacillus amyloliquefaciens FZB42 or Bacillus pumilus, and to pharmaceutical compositions comprising plantazolicin or a salt or an ester thereof. Also provided are methods for producing and using such plantazolicin compounds.

### BACKGROUND OF THE INVENTION

With facile access to low-cost next-generation DNA sequencing technology, there has been a recent surge in genome sequencing. The availability of nearly 2,000 microbial genomes has rekindled interest in the biosynthetic capabilities of bacteria (Challis, G. L. (2008) J Med Chem 51, 2618-2628; Gross, H. (2009) Curr Opin Drug Discov Devel 12, 207-219; Melby et al., (2011) Curr Opin Chem. Biol., 15(3):369-78). Given the status of natural products and their derivatives as the largest source of all medicines, exploring uncharted biosynthetic territory holds vast potential (Newman and Cragg, (2007) J Nat Prod 70, 461-477). One such region of natural product space includes the thiazole/oxazole-modified microcin (TOMM) family (Haft et al., (2010) BMC Biol 8, 70; Lee et al., (2008) Proc Natl Acad Sci USA 105, 5879-5884; Scholz et al. (2011) J Bacteriol 193, 215-224).

Microcins are antibacterial peptides that differ from popular broad-range antibiotics in a variety of ways. One important difference is that microcins target a narrow spectrum of bacteria. As a result, natural human microbial flora will go undisturbed aiding in decreased side effects. A second important difference is that microcins are less likely to be horizontally transferred due to their narrow target spectrum and complex machinery required for synthesis and export, which is often encoded on multiple genes.

Unlike the well-known non-ribosomal peptides and polyketides, TOMMs are derived from inactive, ribosomally synthesized precursor peptides. Each TOMM precursor peptide harbors an N-terminal leader region that serves as the binding site for enzymes that posttranslationally modify a C-terminal core region (Madison et al., (1997) Mol Microbiol 23, 161-168; Mitchell et al., (2009) J Biol Chem 284, 13004-13012). The distinguishing chemical features of a TOMM are heterocycles that derive from cysteine, serine, and threonine residues, which are abundant in the core region of the precursor peptide. During processing by a genetically conserved cyclodehydratase, select cysteines and serine/threonine amino acids undergo peptide backbone cyclization to become thiazoline and (methyl)oxazoline heterocycles. A subset of these are further subjected to a flavin mononucleotide (FMN)-dependent dehydrogenation, which yields the aromatic thiazole and (methyl)oxazole heterocycles. The formation of heterocycles on TOMM precursor peptides is dependent on the presence of a third component, termed the docking protein, whose exact function remains enigmatic (McIntosh, J. A. and Schmidt, E. W., (2010) Chembiochem 11, 1413-1421; Milne et al., (1998) Biochemistry 37, 13250-13261; Milne et al., (1999) Biochemistry 38, 4768-4781). Together, the TOMM cyclodehydratase (C), dehydrogenase (B), and docking protein (D) comprise a functional, heterotrimeric thiazole/oxazole synthetase.

The genes encoding for this synthetase are typically located as adjacent open reading frames in bacterial genomes, making such biosynthetic clusters relatively easy to identify using routine bioinformatic methods (Lee et al., (2008) Proc Natl Acad Sci USA 105, 5879-5884). TOMM biosynthetic clusters often contain ancillary tailoring enzymes that increase the chemical complexity of this natural product family.

Although the unification of the TOMM family of natural products has only recently emerged, the molecular structure and biological function of some TOMMs have long been established. Examples include microcin B17 (DNA gyrase inhibitor), the cyanobactins (eukaryotic cytotoxins), streptolysin S (virulence-promoting cytolysin), and the thiopeptides (ribosome inhibitors) (Melby et al., (2011) Curr Opin Chem. Biol., 15(3):369-78). Bacillus amyloliquefaciens FZB42 is known to produce a plethora of complex small molecules, including bacillaene, difficidin, macrolactin, surfactin, fengycin, bacillomycin D, and bacillibactin (Chen et al., (2007) Nat Biotechnol 25, 1007-1014; Chen et al., (2006) J Bacteriol 188, 4024-4036; Borriss et al., (2010) Int J Syst Evol Microbiol, 61(Pt 8):1786-801).

### SUMMARY OF THE INVENTION

One aspect of the present invention is directed to a plantazolicin-like compound having the structure:

(X¹)—(X²)₅—(X³)₂—(X⁴)₅—(X⁵)ₙ

or a pharmaceutically acceptable salt or ester thereof, wherein X¹ is

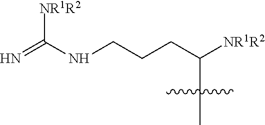

R¹ and R² are each independently hydrogen or lower alkyl; each X² is independently an azole; each X³ is independently a hydrophobic amino acid; each X⁴ is independently an azole or azoline; and each X⁵ is independently an amino acid, wherein n is 1 or 2.

Another aspect of the present invention is directed to a plantazolicin compound having the structure

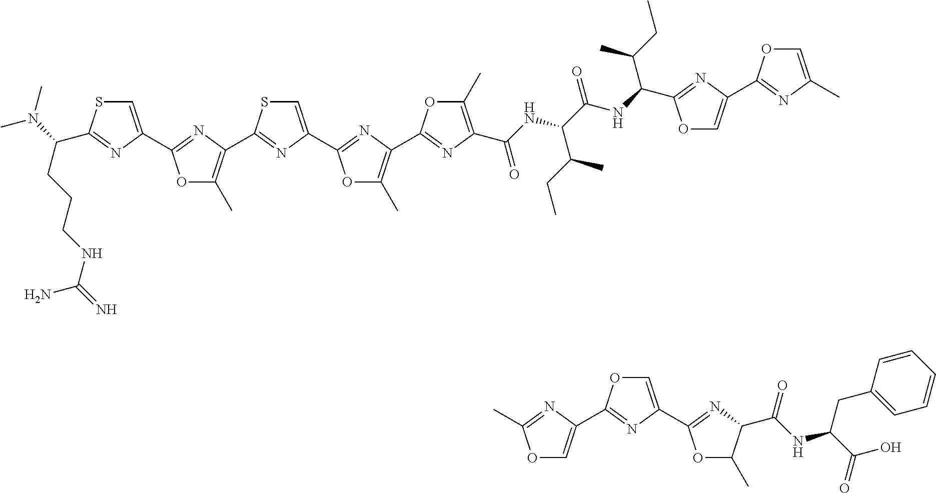

or a pharmaceutically acceptable salt or ester thereof.

It is still another aspect of the present invention to provide a pharmaceutical composition comprising a plantazolicin-like compound described herein or plantazolicin, and a pharmaceutically acceptable carrier.

Yet another aspect of the present invention is a pharmaceutical composition for treating or preventing a Bacillus anthracis infection or a Bacillus cereus infection wherein the therapy comprises administering a pharmaceutical composition disclosed herein to an animal subject in need thereof.

It is another aspect of the present invention to provide a method for treating or preventing a Bacillus anthracis infection or a Bacillus cereus infection, the method comprising administering to a pharmaceutical composition disclosed herein to an animal subject in need thereof.

Among other aspects of the present invention is a method for identifying a plantazolicin-like protein by identifying a bacterial amino acid sequence exhibiting at least 50% amino acid identity to a plantazolicin precursor peptide from Bacillus amyloliquefaciens FZB42; obtaining a post-translationally modified product of the bacterial amino acid sequence; and testing the post-translationally modified product of the bacterial amino acid sequence in a Bacillus anthracis growth inhibitory assay, wherein ability to inhibit the growth of Bacillus anthracis indicates that the bacterial amino acid sequence encodes a plantazolicin-like protein.

Still other aspects of the present invention are directed to methods for producing the plantazolicin. In one aspect, plantazolicin is produced by growing Bacillus amyloliquefaciens FZB42 cells in culture; collecting the Bacillus amyloliquefaciens FZB42 cells, thereby obtaining the harvested Bacillus amyloliquefaciens FZB42 cells; obtaining a crude plantazolicin extract from the harvested Bacillus amyloliquefaciens FZB42 cells; and purifying the plantazolicin compound from the crude plantazolicin extract.

In another aspect, plantazolicin is produced by growing Bacillus pumilus cells in culture; collecting the Bacillus pumilus cells, thereby obtaining the harvested Bacillus pumilus cells; obtaining a crude plantazolicin extract from the harvested Bacillus pumilus cells; and purifying the plantazolicin compound from the crude plantazolicin extract.

Other objects and features will be in part apparent and in part pointed out hereinafter.

### BRIEF DESCRIPTION OF THE DRAWINGS

FIG. 1 depicts mass spectrometry-based structural elucidation of PZN. A, After biosynthetic processing, the final chemical structure of PZN features N^{α},N^{α}-dimethylArg (green), two thiazoles (red), seven (methyl)oxazoles (blue), and one methyloxazoline (brown). The numbering scheme used for each original residue is given at the top of the figure. After treatment with mild acid, azoline heterocycles undergo hydrolytic ring opening to the original amino acid (in this case, Thr). Stereochemical configuration is assumed to be identical to the ribosomally produced peptide precursor. B, CID spectrum of PZN (m/z 1336) acquired by LTQ-FT-MS. C, Same as B, except the parental ion analyzed was hydrolyzed PZN (m/z 1354). *Denotes ions resulting from the loss of guanidine that localize the site of dimethylation to the α-amine of Arg. Localization to Arg is further supported by loss of N^{α},N^{α}-dimethylArg (m/z 1180). **Denotes ions indicating that the sole azoline moiety of PZN is derived from the most C-terminal Thr residue. All masses are given in Daltons (Da) and represent the singly-charged ion. For proposed structures of the daughter ions, see FIGS. 7-8.

FIG. 2 shows the effect of oxygenation during fermentation on the production of PZN. Cultivation at both high (biofermentor) and low (flasks) oxygen levels (see methods in Example 2) were grown for 24 hours at 37° C. All samples were extracted and subjected to chromatography using an identical procedure. In all panels, vertical lines were drawn at 14.7, 19.9, and 20.5 min. A, UV chromatogram (Abs 266 nm) of FZB42 strain RSpMarA2 extract from high and low oxygen fermentation.

B, Same as A except the trace is the total ion chromatogram (TIC). C, Extracted ion chromatogram (EIC) of m/z 1336, 1338, and 1354 from a low oxygen fermentation.
D, Same as C except under high oxygenation conditions.

FIG. 3 shows an assessment of PZN antibiotic activity. A, The minimum inhibitory concentration (MIC) of HPLC-purified PZN was measured against a panel of Gram-positive human pathogens. Values reported were the concentration of PZN that inhibited 99% of the bacteria growth in a microbroth dilution bioassay. *Due to separation difficulties, dihydroPZN was supplied as a 1:2:2 mixture of non-, mono-, and dihydrolyzed species (m/z 1338, 1356, 1374). B, PZN activity in an agar disk diffusion bioassay against B. anthracis Sterne. Upper left disk, 8 μg kanamycin control (positive); upper right, solvent control (negative); lower disk, 100 μg PZN (200 μg gave a similar inhibition diameter). C, Visual appearance of live B. anthracis Sterne treated with a solvent control by DIC microscopy. D, Same as panel C, except cells were treated with 4 μg/mL PZN. Scale bar is the same for panel C.

FIG. 4 depicts the PZN biosynthetic gene clusters. A, Open-reading frame diagram showing the genetic organization of PZN clusters, which form a subclass of thiazole/oxazole-modified microcins (TOMMs). Gene designations and predicted functions are color coded in the provided legend. B, PZN precursor peptide (PznA) alignment. Shown in purple are conserved residues within the N-terminal leader region. *Denotes the PznA leader peptide cleavage site, which is known for BamA and BpumA but predicted for the others. Color-coding indicates the posttranslational modification found at each residue in the BamA core region (other precursor peptide modifications are extrapolated from the known structure of PZN from FZB42). The conserved Arg (green) undergoes two methylation events to yield N^{α},N^{α}-dimethylArg. Cys (red) are converted to thiazoles while all blue residues become (methyl)oxazoles. The most C-terminal cyclizable position in BamA (brown, Thr) is left as an azoline heterocycle. Abbrev.: Bam, Bacillus amyloliquefaciens FZB42; Bpum, Bacillus pumilus ATCC 7061; Cms, Clavibacter michiganensis subsp. sepedonicus; Cur, Corynebacterium urealyticum DSM 7109; Blin, Brevibacterium linens BL2. Bam and Bpum are Firmicutes (Gram-positive, low % GC genome), while the other three species are Actinobacteria (Gram-positive, high % GC genome).

FIG. 5 shows the FTICR-MS of PZN (m/z 1336, broadband and CID spectrum of 2+ charge state). A, Broadband spectrum of HPLC-purified PZN on a linear ion trap MS (11 Tesla LTQ-FT). Visible are the singly and doubly charged positive ions of PZN. Due to the high mass accuracy of FT-MS (<5 ppm error) and the known sequence of the precursor peptide (1, 2), the molecular formula of PZN was deduced from this mass measurement. The PZN molecular formula (neutral species) is C₆₃H₆₉N₁₇O₁₃S₂ (monoisotopic mass, 1335.4702; error, 0.15 ppm). This formula required that 9 out of the 10 heterocyclizable residues were converted to the azole heterocycle and the remaining residue was left at the azoline oxidation state. Also, this formula required two methylation events (consistent with earlier deletion studies) and leader peptide cleavage after Ala-Ala (see FIG. 4b). B, Collision induced dissociation (CID) spectrum of m/z 668.7 (PZN²⁺). The fragmentation pattern of PZN in the doubly charged state is markedly different than that of the singly charged species shown in FIG. 1B. The amino acid sequence for the PZN precursor peptide from B. amyloliquefaciens FZB42 (BamA) is color-coded by posttranslational modification as follows: N^{α},N^{α}-dimethylarginine (green), thiazoles (red), methyloxazoles and oxazoles (blue), and methyloxazoline (brown). Identified fragment ions are also plotted onto the BamA precursor sequence. The most diagnostic peaks for localizing posttranslational modifications resulted from Ile-Ile cleavage (green and brown mass peaks). These ions demonstrate that both methylation events are on the N-terminal fragment and that the sole azoline moiety is on the C-terminal fragment. *Fragment ions with the azoline as the most C-terminal moiety spontaneously decompose, supporting the assignment of the C-terminal Thr as being converted to methyloxazoline in PZN (assigned in FIG. 7). Under the CID conditions employed, most peptides fragment at the amide bond. The first step in TOMM biosynthesis, cyclodehydration, removes an amide bond from the peptide backbone. **Contiguous heterocycles thus preclude the formation of a complete series of y⁺ and b⁺ ions and result in a CID spectrum that is featureless from m/z ˜710-1100. One non-amide cleavage is noted between arginine and cysteine (highest mass ion in the spectrum), which permits the methyl groups to both be localized to arginine. ̂Internal fragments (assigned in FIG. 7).

FIG. 6 depicts the UV-Vis spectrum of HPLC-purified PZN in DMSO acquired on a Nanodrop 2000. The instrument was blanked on DMSO, which has a UV cut-off of approximately 245 nm. The extinction coefficient for PZN in DMSO is ε₂₆₀=560 M⁻¹ cm⁻¹. The λₘₐₓ in 80% acetonitrile/water is 266 nm.

FIG. 7 shows the fragmentation map for PZN (m/z 1336). Fragmentation pathways in ion trap mass spectrometers are typically not sequential and most often result from the product of a single cleavage event. Thus, the arrows in this diagram are for illustrative purposes and are not meant to represent an actual pathway. The most revealing masses are boxed, in addition to the parent ion (PZN). The m/z 1277 structure (green text) results from the dissociation of guanidine from PZN and permits the localization of both methyl groups to the N-terminus of PZN. The m/z 1145 structure (brown text) results from the loss of the C-terminal Phe residue and CO. In conjunction with selective hydrolysis studies, m/z 1145 and the subsequent azoline decomposition ions localize the sole azoline as the C-terminal Thr residue. Further, there are many examples of neutral loss of acetaldehyde (C₂H₄O, exact mass=44.0262; not to be confused with loss of carbon dioxide, exact mass=43.9898; >800 ppm difference).

FIG. 8 shows the fragmentation map for hydrolyzed PZN (m/z 1354). Unlike their aromatic azole counterparts, azoline heterocycles are hydrolytically unstable in mild acid and mild base. Selective acidic hydrolysis of PZN was performed to convert the sole azoline heterocycle back to the original amino acid. This reinstated an amide bond that can be located by subsequent MSⁿ analysis. As mentioned in FIG. 7, the arrows are for illustration purposes only and not meant to indicate that the ions fragment following the path shown. Ions that are duplicative with those given in FIG. 7 are not replicated here. There were no cases of neutral loss of acetaldehyde following methyloxazoline hydrolysis to threonine. This implies that loss of acetaldehyde (formation of azirine) is specific to methyloxazolines under the CID conditions that were employed. The loss of C₂H₄O was possible from hydrolyzed (Thr-containing) PZN, but only via dehydration (could also be α/β, drawn as (β/γ) and subsequent loss of acetylene. Other fragment ions of interest in this map confirmed the site of dimethylation to be the N-terminal amine.

FIG. 9 shows the CID spectra for deguanidinated PZN (m/z 1277) and deguanidinated hydrolyzed PZN (m/z 1295). MS³ collision induced dissociation (CID) spectra for A, deguanidinated PZN (m/z 1277) and B, deguanidinated hydrolyzed PZN (m/z 1295). **Indicates loss of acetaldehyde (44.0262 Da) from methyloxazoline (1277−44=1233; 1194−44=1150). Note that this is only possible in panel A, where an intact heterocycle is found. The ions at m/z 575, 1150, and 1194 demonstrate that the Arg was dimethylated on the amino terminus. Structural assignments are given for the fragments of deguanidinated PZN and deguanidinated hydrolyzed PZN in FIGS. 7 and 8, respectively.

FIG. 10 depicts the N-terminal labeling of PZN and desmethylPZN using NHS-biotin. MALDI-TOF-MS results of NHS-biotin labeling for A, PZN (m/z 1336) and hydrolyzed PZN (m/z 1354) and B, desmethylPZN (m/z 1308) and hydrolyzed desmethylPZN (m/z 1326). Abbreviation: desmethylPZN, dmPZN. Red traces are samples that included the NHS-biotin reagent while black traces are from control reactions that lacked NHS-biotin. Labeling was only observed with desmethylPZN, as indicated by the new species at m/z 1534 and 1552. Addition of biotin gives a net mass increase of 226 Da (C₁₀H₁₄N₂O₂S). Specific labeling reactions are given in the methods section.

FIG. 11 shows the ¹H-¹H-gCOSY of PZN. Assigned correlations are drawn on the structure of PZN as thickened bonds. The brown circles indicate correlations deriving from the methyloxazoline protons (shown as brown bonds in structure). The red asterisk indicates that in the 1D-¹H-spectrum, the signal from water was suppressed. This signal was not suppressed for the 2D experiment.

FIG. 12 shows the ¹H-¹H-TOCSY of PZN. Assigned correlations are drawn on the structure of PZN as thickened bonds. The brown circles indicate correlations deriving from the methyloxazoline protons (shown as brown bonds in structure). The red asterisks on the 1D spectra indicate the signal from water suppression. This signal was also suppressed for the 2D experiment.

FIG. 13 depicts the ¹H-¹³C-gHMBC of PZN. Assigned correlations are drawn on the structure of PZN as red arrows. The green arrows/circles indicate correlations that localize the posttranslational methyl groups to the N-terminus. The brown arrows/circles indicate correlations that demonstrate the azoline is methyloxazoline.

FIG. 14 shows the predicted isotope pattern for PZN (m/z 1336). The average mass is slightly heavier than the first isotope mass. This figure was generated using iMass version 1.1 (freeware written by Urs Roethlisberger).

FIG. 15 shows the effect of oxygen levels during fermentation on the production of PZN. ESI-MS at selected time points from LCMS analysis (UV, TIC, EIC) is shown in FIG. 2. A, Under low oxygen conditions, PZN (m/z 1336) is the only species present in the 19.9 min elution. B, Under an oxygen saturated fermentation, PZN is found in the 20.5 min elution. C, As expected from the EIC's shown in FIG. 2, high oxygen fermentation yields an additional compound eluting at 14.7 min consistent with dihydroPZN (dhPZN, m/z 1338). The earlier elution of dhPZN relative to PZN is in agreement with azolines being more hydrophilic and basic than azoles (azoles are not protonated with 0.1% formic acid). Right insets for all panels show a zoomed-in spectrum to highlight the isotopic pattern of the singly charged PZN species.

FIG. 16 depicts the localization of second azoline heterocycle on dihydroPZN (dhPZN). A, CID spectrum of dhPZN (m/z 1338) acquired using LTQ-FT-MS. The heavier fragment ions are identical to those shown in FIG. 1, with the exception of each fragment being 2 Da heavier. The gray box depicts a zoomed-in region shown in panel B. Brown boxes highlight two ions demonstrating that an azoline heterocycle exists on each side of the Ile-Ile. The location of the C-terminal azoline was localized to the most C-terminal Thr. The location of the N-terminal azoline is likely to be the Thr adjacent to Ile due to similar sterics/electronics. However, the precise position cannot be concluded from this spectrum. B, Zoomed-in region from panel A (gray box). Diagnostic ions are boxed in gray and their respective (predicted) structures are drawn in the right margin.

FIG. 17 depicts the effect of oxygen levels during fermentation on the production of desmethylPZN: UV, TIC, and EIC traces of desmethylPZN (m/z 1308), dihydrodesmethylPZN (m/z 1310), and hydrolyzed desmethylPZN (m/z 1326). In each case presented, the low oxygen samples were prepared by shake flask fermentation of B. amyloliquefaciens strain RS33 (pznL deletion, desmethylPZN producer) in 2 L of LB in 6 L flasks. High oxygen samples were prepared using a biofermentor with 5 L/min air input. Both cultures were grown for 24 h at 37° C. All samples were extracted in an identical fashion and subjected to identical chromatographic procedures (analytical C₁₈—HPLC) as described in the methods. In all panels, vertical lines are drawn at 14, 17, and 21 min. A, UV chromatogram (Abs 272 nm) of RS33 extract from high and low oxygen fermentation. This trace shows that more chromophores absorbing light at 272 nm are produced under high oxygen conditions. B, Same as A except the trace is the total ion chromatogram (TIC). C, Extracted ion chromatogram (EIC) of m/z 1308, 1310, and 1326 from low oxygen fermentation. Under these conditions, the majority species is desmethylPZN (1308) with trace amounts of hydrolyzed desmethylPZN (1326). The 1310 trace that appears to “coelute” with 1308 at 17 min is actually the second isotope peak of 1308, not dihydrodesmethylPZN (see FIG. 14). D, Same as C except under high oxygenation conditions. The peaks at 14 and 16 min contain primarily dihydrodesmethylPZN (m/z 1310) while the peaks at 17 and 21 min contain primarily desmethylPZN (m/z 1308). The species eluting at 14 and 16 min are suspected to be regioisomers, as are the species eluting at 17 and 21 min. ESI-MS data at these selected time points are shown in FIG. 18.

FIG. 18 shows the effect of oxygen levels during fermentation on the production of desmethylPZN. ESI-MS at selected time points from LCMS analysis (UV, TIC, EIC) is shown in FIG. 17. A, Under low oxygen conditions, hydrolyzed desmethylPZN (m/z 1326) is visible in the 14 min elution. B, As expected from the EIC's shown in FIG. 17, the 14 min elution is dominated by dihydrodesmethylPZN (m/z 1310) at the 14 min elution. C, Low oxygen fermentation and an elution of 17 min yields exclusively desmethylPZN (m/z 1308). As indicated by the ion purity and signal to noise ratio in this spectrum, relative to the other panels, desmethylPZN was a majority product and easily separated under the conditions employed. D, Same as C but high oxygen conditions led to the production of a mixture of desmethylPZN and dihydrodesmethylPZN (ratio ˜60:40, respectively). E, At 16 min under high oxygen conditions, 1310 is the majority species produced, consistent with azolines being more hydrophilic than azoles. Right insets for panels C-E show a zoomed-in spectrum to highlight the isotopic pattern of the singly charged desmethylPZN species. The ratio of intensities given in FIG. 14 applies.

FIG. 19 shows the similarity/identity matrix of related (PZN-producing) biosynthetic proteins. Shown in yellow are amino acid identity scores obtained by pairwise alignment using ClustalW2, which includes the standard parameters for gap penalties. In blue are the corresponding amino acid percent similarity values, obtained by recording the ratio of similar amino acids to the full protein sequence after alignment (no gap penalties). Abbreviations: PznJ, required biosynthetic protein of unknown function; PznC, cyclodehydratase; PznD, docking protein; PznB, FMN-dependent dehydrogenase; PznE, suspected leader peptidase; and PznL, SAM-dependent methyltransferase. Abbreviations used are derived from the genus and species name for each organism. Bam, Bacillus amyloliquefaciens FZB42; Bpum, Bacillus pumilus ATCC 7061; Cms, Clavibacter michiganensis subsp. sepedonicus; Cur, Corynebacterium urealyticum DSM 7109; and Blin, Brevibacterium linens BL2. Bam and Bpum are Firmicutes, while the other three species are Actinobacteria.

FIG. 20 depicts the PZN production from Bacillus pumilus ATCC 7061. Cells were grown in an identical fashion to B. amyloliquefaciens. A, The cell surface metabolites were extracted with methanol, dried, concentrated, and separated on a preparative C₁₈—HPLC column with UV monitoring at 266 nm (λₘₐₓ for PZN). B, The 22-min (top), 23-min (middle), and 24-min (bottom) fractions from HPLC purification were concentrated and spotted on to the MALDI target with sinapic acid. In the earliest fraction, m/z 1354 (hydrolyzed PZN) is visible. In the latter two fractions, m/z 1336 (PZN) is readily identified and pooled for further analysis. C, HPLC purified PZN from B. pumilus was subjected to high-resolution MS (LTQ-FT-MS), which verified the molecular formula to be consistent with PZN within the mass accuracy of the instrument (<5 ppm). D, CID spectrum obtained upon isolation of the singly charged (m/z 1336) precursor ion. This data is analogous to FIG. 1B (PZN from B. amylo. RSpMarA2). E, CID spectrum obtained upon isolation of the doubly charged (m/z 668) precursor ion. This data is analogous to FIG. 5B (PZN from B. amylo. RSpMarA2). Different instrumental settings had to be employed to visualize the PZN ions, which were less abundant than from the B. amylo. overproducer (RSpMarA2) and required summing over many scans. An unidentified contaminant and instrumental noise account for the ions between m/z 750-1100.

FIG. 21 depicts the plantazolicin gene cluster. A, FZB42 PZN gene cluster (9892 bp) and amino acid sequence of the precursor peptide. (−) marks a putative leader peptide processing site. B, Proposed function of individual PZN genes. Upon deletion of pznF and pznl, cpd1336 (PZN) was detected by mass spectrometry. Deletion of pznL resulted in desmethyl PZN (m/z=1308 Da) while individual inactivation of all other tested genes (pznABCJ) did not produce PZN.

Corresponding reference characters indicate corresponding parts throughout the drawings.

### DEFINITIONS AND ABBREVIATIONS

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

The term “expression cassette” is used to define a nucleotide sequence containing regulatory elements operably linked to a coding sequence that result in the transcription and translation of the coding sequence in a cell.

The term “plasmid” as used herein, refers to an independently replicating piece of DNA. It is typically circular and double-stranded.

As used herein, Bacillus anthracis spore (or anthrax spore) is a small reproductive body produced by B. anthracis bacteria. Such spores do not form normally during active growth and cell division. Rather, their differentiation begins when a population of vegetative cells passes out of the exponential phase of growth, usually as a result of nutrient depletion.

“Preventing” a disease refers to inhibiting the full development of a disease, for example preventing development of anthrax disease. Prevention of a disease does not require a total absence of infection. For example, a decrease of at least 50% can be sufficient.

“Treatment” or “treating” refers to a therapeutic intervention that ameliorates a sign or symptom of a disease or pathological condition, such a sign or symptom of anthrax disease (e.g., fever, ulcers, swollen lymph nodes, skin blisters). Treatment can also induce remission or cure of a condition, such as anthrax disease, including inhalational anthrax, gastrointestinal anthrax, oropharyngeal anthrax and cutaneous anthrax.

The term “pharmaceutically acceptable salt” refers to the relatively non-toxic, inorganic and organic acid addition salts of compounds of the present invention. These salts can be prepared in situ in the administration vehicle or the dosage form manufacturing process, or by reacting a purified compound of the invention in its free base form with a suitable organic or inorganic acid, and isolating the salt thus formed during subsequent purification. Representative salts include the hydrobromide, hydrochloride, sulfate, bisulfate, malate, citrate, flurbiprofen, ketoprofen, loxoprofen, diclofenac, etodolac, indomethacin, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, laurylsulphonate salts and the like. (See, for example, Berge et al. (1977) “Pharmaceutical Salts,” J. Pharm. Sci. 66: 1-19).

“PZN” as used herein is an abbreviation for plantazolicin.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the comparison window, and multiplying the result by 100 to yield the percentage of sequence identity.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

The term “similarity,” or percent “similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Such sequences are then said to be “substantially similar.” Optionally, this identity exists over a region that is at least about 50 amino acids in length, or more preferably over a region that is at least about 100 to 500 or 1000 or more amino acids in length.

### DESCRIPTION OF THE PREFERRED EMBODIMENTS

The present invention is directed to novel plantazolicin-like compounds, which are highly discriminating antibiotics (i.e., they are narrow-spectrum antibiotics in that they are active against a selected group of bacterial types and used for the specific infections arising from these bacterial types). The plantazolicin-like compounds structurally belong to a family of thiazole/oxazole-modified microcins (TOMMs).

One aspect of the present invention is directed to a plantazolicin-like compound having the structure: (X¹)—(X²)₅—(X³)₂—(X⁴)₅—(X⁵)ₙ or a pharmaceutically acceptable salt or ester thereof, wherein X¹ is

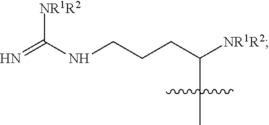

R¹ and R² are each independently hydrogen or lower alkyl; each X² is independently an azole; each X³ is independently a hydrophobic amino acid; each X⁴ is independently an azole or azoline; and each X⁵ is independently an amino acid, wherein n is 1 or 2.

In some embodiments, X² is selected from the group consisting of: pyrazole, imidazole, thiazole, oxazole, isoxazole, isothiazole, pyrrole, triazole, tetrazole, and pentazole, and is preferably thiazole or oxazole.

In some instances, X³ is selected from the group consisting of alanine (Ala), valine (Val), isoleucine (Ile), leucine (Leu), methionine (Met), phenylalanine (Phe), tyrosine (Tyr), and tryptophan (Trp). Preferably, X³ is isoleucine, phenylalanine, or tryptophan.

In some embodiments, X⁴ is an azole selected from the group consisting of pyrazole, imidazole, thiazole, oxazole, isoxazole, isothiazole, pyrrole, triazole, tetrazole, and pentazole, or an azoline selected from the group consisting of pyrazoline, imidazoline, thiazoline, oxazoline, isoxazoline, isothiazoline, pyrroline, triazoline, tetrazoline, and pentazoline. X⁴ is preferably is thizole, oxazole or oxazoline.

In some instances, X⁵ is selected from the group consisting of phenylalanine, tyrosine and tryptophan. In an exemplary preferred embodiment, X⁵ is phenylalanine.

Preferably, n is 1.

Another aspect of the present invention is directed to plantazolicin having the structure

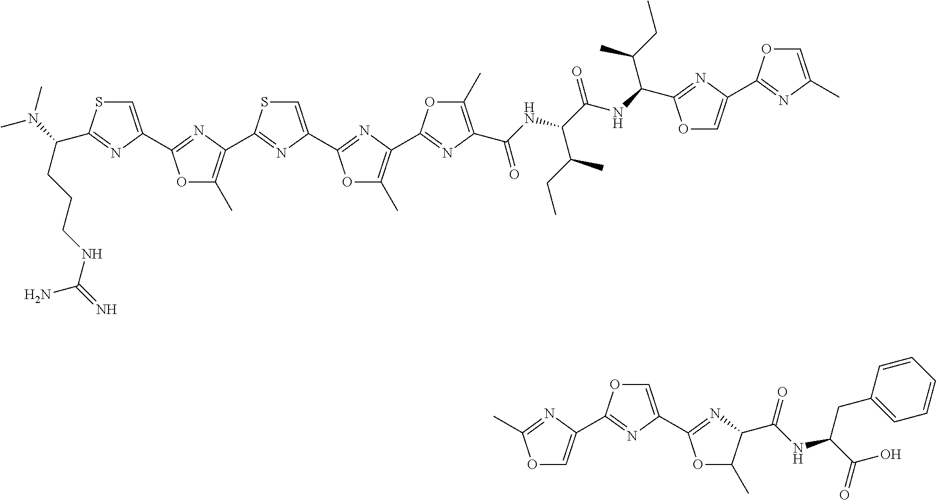

or a pharmaceutically acceptable salt or ester thereof.

The plantazolicin-like compound described above can be synthesized by any methods known in the art, such as by total chemical synthesis, semi-synthesis or bacterial strain bioengineering.

In one embodiment, plantazolicin is isolated from Bacillus amyloliquefaciens FZB42. Bacillus amyloliquefaciens FZB42 is a gram-positive, plant-growth promoting bacterium with a large capacity to produce secondary metabolites with antimicrobial activity.

Plantazolicin, belonging to a class of TOMM molecules is produced from a small precursor peptide that is posttranslationally modified to contain thiazole and (methyl)oxazole heterocycles. These rings are derived from Cys and Ser/Thr through the action of a trimeric ‘BCD’ synthetase complex, which consists of a cyclodehydratase (C), dehydrogenase (B), and a docking protein (D).

In general, during TOMM biosynthesis, the precursor peptide is bound by the BCD synthetase complex through specific motifs within the N-terminal leader sequence. After substrate recognition, heterocycles are synthesized on the C-terminal core peptide over two enzymatic steps. The first is carried out by a cyclodehydratase, which converts Cys and Ser/Thr residues into the corresponding thiazolines and (methyl)oxazolines. A dehydrogenase then oxidizes the ‘azoline’ rings to yield ‘azole’ rings [thiazoles and (methyl)oxazoles], resulting in a net loss of 20 Da. The completion of TOMM biosynthesis includes the incorporation of ancillary modifications (e.g. dehydrations, methylations, macrocyclization, etc.), and leader peptide proteolysis. In many cases, the fully mature TOMM natural product is then actively exported from the cell through the use of an ABC transport system.

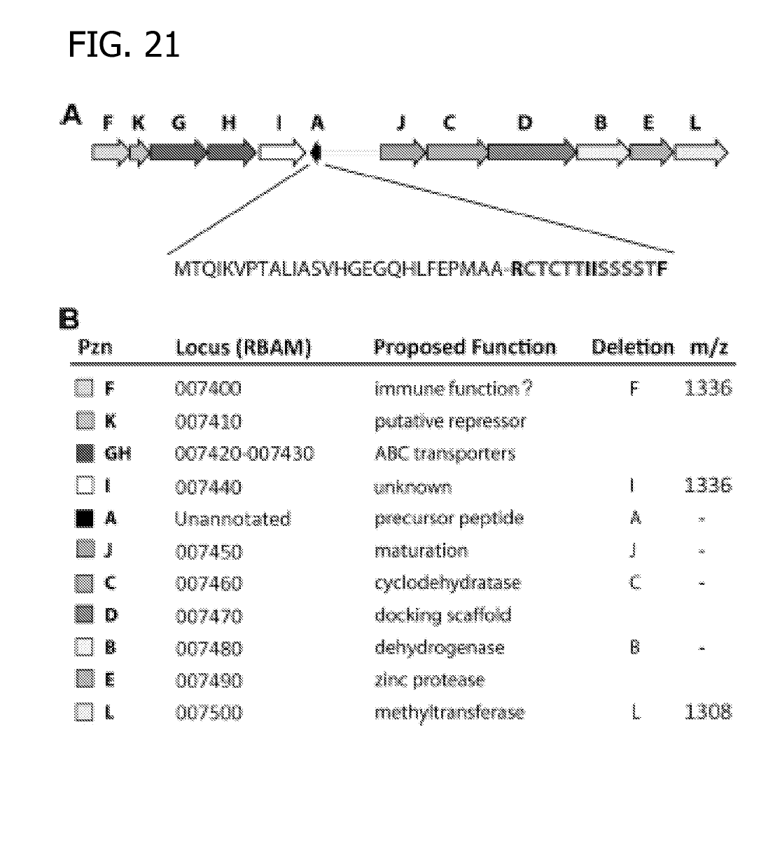

The PZN biosynthetic 12-gene cluster spans nearly 10 kb of the FZB42 chromosome as shown in FIG. 21. Furthermore, as shown in the examples, plantazolicin biosynthetic genes are transcribed into two polycistronic mRNAs (pznFKGHI and pznJCDBEL) and a monocistronic mRNA for pznA. The gene coding for a precursor peptide was identified by a manual ORF search and found to be encoded between pznI (RBAM_{—}007440) and pznJ (RBAM_{—}007450) in the opposite direction. Although it is not annotated, this ORF (pznA) bears a robust Shine-Dalgarno sequence, AGGAGG, which lies 8 base pairs upstream of an AUG start codon. The C-terminal region, also known as the core peptide was found to be rich in residues that can be enzymatically cyclized to thiazoles and (methyl)oxazoles (2 Cys, 4 Thr, and 4 Ser).

The first operon (pznFKGHI) consists of genes predicted to be involved in immunity, regulation, and transport (FIG. 21). The product of pznK (RBAM_{—}007410) is related to homodimeric repressor proteins of the ArsR family. While not being bound to a particular theory, this protein possibly regulates the expression of other PZN genes through an unexplored mechanism. The second operon (pznJCDBEL) harbors the genes encoding for the enzymes responsible for converting the inactive PznA precursor peptide into the mature, bioactive natural product. A summary of the putative function of the members of the PZN gene cluster in B. amyloliquefaciens FZB42 is given in FIG. 21.

Based on sequence alignment with other known TOMM biosynthetic cluster genes, PznC is related to the TOMM cyclodehydratase and believed to act as one. PznD is highly similar to SagD from the SLS biosynthetic cluster and is termed the docking scaffold protein, while PznE is believed to be a leader peptidase.

In one embodiment, plantazolicin is produced from a precursor peptide having the amino acid sequence MTQIKVPTALIASVHGEGQHLFEPMAARCTCTTIISSSSTF (SEQ ID NO: 1). In another embodiment, the core region of the precursor peptide has the sequence of RCTCTTIISSSSTF (SEQ ID NO: 2).

In another embodiment, the present invention is directed to a method for producing plantazolicin by growing Bacillus amyloliquefaciens FZB42 cells in culture; collecting the Bacillus amyloliquefaciens FZB42 cells, thereby obtaining the harvested Bacillus amyloliquefaciens FZB42 cells; obtaining a crude plantazolicin extract from the harvested Bacillus amyloliquefaciens FZB42 cells; and purifying plantazolicin from the crude plantazolicin extract. In other embodiments, any other Bacillus amyloliquefaciens strain, whether mutated or not that is capable of producing PZN can be used in this method.

Using a protein BLAST search, it was discovered that thiazole/oxazole synthetase proteins (PznBCD) from B. pumilus (protein IDs: EDW22765.1, EDW22903.1, and EDW23125.1, respectively) demonstrated a significant degree of amino acid identity to those from FZB42 (PznB, 77%; PznC, 63%, and PznD, 82%). Moreover, the PZN genes from B. pumilus were found to be clustered and in identical order to that found in FZB42. Similarly to FZB42, B. pumilus is a plant saprophyte that produces an array of antibacterial and antifungal natural products. Furthermore, the PznA core peptide sequences from FZB42 and B. pumilus (unmarked, located between EDW23486.1 and EDW22932.1) were found to be 100% identical, and plantazolicin isolated from B. pumilus was identical to FZB42-isolated PZN. Hence, it is another embodiment of the present invention to provide plantazolicin isolated from Bacillus pumilus.

Accordingly, it is another embodiment of the present invention to provide a method for producing plantazolicin, the method comprising growing Bacillus pumilus cells in culture; collecting the Bacillus pumilus cells, thereby obtaining the harvested Bacillus pumilus cells; obtaining a crude plantazolicin extract from the harvested Bacillus pumilus cells; and purifying plantazolicin from the crude plantazolicin extract.

In some embodiments related to the methods for producing PZN, cells, either B. amyloliquefaciens or B. pumilus, are grown in flasks. In other embodiments, the cells are grown in biofermentors. This is especially desirable when larger quantities of plantazolicin are being produced. One of ordinary skill in the art can readily determine culture media and growth conditions necessary to produce plantazolicin in particular cells. Some of the exemplary conditions for both B. amyloliquefaciens and B. pumilus are shown in the examples.

The inventors also discovered that low and high oxygen levels used during cell growth, i.e. fermentation affected the production of PZN and its derivative metabolites. In particular, there was more PZN produced compared to metabolites during low oxygenation, whereas high oxygenation resulted in greater production of metabolites, which were either less active (dihydroPZN) or inactive (desmethylPZN) compared to PZN. Hence, in some embodiments, the step of growing cells for production of PZN is performed under low oxygen conditions. Low oxygenation refers to conditions such as regular growth of bacteria in flasks (i.e., at oxygen levels present in air), whereas high oxygenation refers to oxygen supplementation, such as by supplying air at a rate of 5 L/min, the air being saturated in oxygen at a rate of approximately 1 L/min.

Cells are next harvested using any of the methods known in the art. In some embodiments, cells are harvested by centrifugation or by any other method used in the art. One or more centrifugation steps can be performed in order to obtain most of the cells. Following the harvesting step, a crude PZN extract is obtained from the harvested cells. In some embodiments, the crude PZN extract is obtained by performing a non-lytic, methanolic extraction of the cellular surface of the harvested cells. Other solvents used for extraction can readily be determined by a skilled artisan. Exemplary conditions are shown in the examples, and a skilled artisan can readily determine other conditions suitable for crude PZN extraction.

A crude plantazolicin extract is next subjected to a purification step, which allows for separation of plantazolicin from other components in the extract. In some embodiments of the present invention, plantazolicin is purified by high performance liquid chromatography (HPLC). One particularly useful method of HPLC is reverse phase HPLC.

As shown in the examples and Table 3, plantazolicin was tested for growth inhibitory activity towards a wide range of bacteria. In total, 18 strains from 16 distinct species were assayed for susceptibility to PZN (Table 3). It was determined that PZN exhibited activity primarily towards Bacillus sp., including B. subtilis. PZN exhibited no activity against any tested Gram-negative organisms. To further define the selectivity within the Gram-positive organisms, the scope of PZN activity towards a panel of ubiquitous human pathogens was evaluated, including methicillin-resistant Staphylococcus aureus (MRSA), vancomycin-resistant Enterococcus faecalis (VRE), Listeria monocytogenes, Streptococcus pyogenes, and Bacillus anthracis strain Sterne (a surrogate for the BSL3 pathogen, which lacks the poly-D-glutamic acid capsule). Plantazolicin exhibited a potent growth inhibition of B. anthracis, whereas all other species were unaffected by PZN (with the exception of S. pyogenes, which was only inhibited by very high concentrations of PZN). The action of PZN upon B. anthracis was shown to be bactericidal, as described in the examples. Accordingly, a plantazolicin-like compound or plantazolicin can be used to inhibit growth of Bacillus species, which is useful for treating infections caused by Bacillus bacteria susceptible to PZN. By way of example and not of limitation, a plantazolicin-like compound or plantazolicin can be used to treat B. cereus infection, which causes food poisoning in humans. In particular, due to a potent bactericidal effect on B. anthracis, a plantazolicin-like compound or plantazolicin can be used as an effective, highly specific highly discriminating antibiotic against anthrax infections.

Accordingly, it is an embodiment of the present invention to provide a pharmaceutical composition comprising a plantazolicin-like compound, plantazolicin or a pharmaceutically acceptable salt or ester thereof and a pharmaceutically acceptable carrier. A plantazolicin-like compound, plantazolicin or a pharmaceutically acceptable salt or ester thereof can be formulated as a pharmaceutical composition prior to administering to an animal subject, according to techniques known in the art. Pharmaceutical compositions of the present invention are characterized as being at least sterile and pyrogen-free. Methods for preparing pharmaceutical compositions of the invention are within the skill in the art, for example as described in Remington's Pharmaceutical Science, 17th ed., Mack Publishing Company, Easton, Pa., (1985).

The present pharmaceutical formulations comprise a plantazolicin-like compound, plantazolicin or a pharmaceutically acceptable salt or ester thereof (e.g., 0.1 to 90% by weight) mixed with a pharmaceutically acceptable carrier. Suitable physiologically acceptable carriers are water, buffered water, saline solutions (e.g., normal saline or balanced saline solutions such as Hank's or Earle's balanced salt solutions), 0.4% saline, 0.3% glycine, hyaluronic acid and the like.

The pharmaceutical composition of the present invention can be administered orally, nasally, parenterally, intrasystemically, intraperitoneally, topically (as by drops or transdermal patch), bucally, sublingually or as an oral or nasal spray. In one preferred embodiment, the pharmaceutical composition of the present invention is administered orally. In another preferred embodiment, the pharmaceutical composition is given intravenously. In still another preferred embodiment, the pharmaceutical composition is given subcutaneously or intramuscularly.

A pharmaceutical composition of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

In some cases, to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle. Prolonged absorption of the injectable pharmaceutical form can be brought about by the inclusion of agents which delay absorption such as aluminum monostearate and gelatin.

Solid dosage forms for oral administration include, but are not limited to, capsules, tablets, pills, powders, and granules. In such solid dosage forms, the active compounds are mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents. Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents. The compositions can also release the active ingredient(s) only, or preferentially, in a certain part of the intestinal tract, optionally, in a delayed manner. Examples of embedding compositions which can be used include polymeric substances and waxes.

The pharmaceutical compositions of the present invention can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying agents, suspending agents, sweetening, flavoring, and perfuming agents.

Suspensions, in addition to a plantazolicin-like compound or plantazolicin, can contain suspending agents such as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Alternatively, the composition can be pressurized and contain a compressed gas, such as nitrogen or a liquefied gas propellant. The liquefied propellant medium and indeed the total composition are preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example calcium chloride, calcium ascorbate, calcium gluconate or calcium lactate).

One of ordinary skill in the art will appreciate that effective amounts of the agents of the invention can be determined empirically and can be employed in pure form or, where such forms exist, in pharmaceutically acceptable salt, ester or prodrug form.

The pharmaceutical composition comprising a plantazolicin-like compound or plantazolicin can be administered to a patient in order to prevent and/or treat anthrax infection. It will be understood that, when administered to a human patient, the total daily usage of the plantazolicin compound or composition of the present invention will be decided by the attending physician within the scope of sound medical judgment, and when administered to an animal, will be determined by a veterinarian. The specific therapeutically effective dose level for any particular patient will depend upon a variety of factors: the type and degree of the cellular or physiological response to be achieved; activity of the plantazolicin compound; the age, body weight, general health, sex and diet of the patient; the time of administration, route of administration, and rate of excretion of the agent; the duration of the treatment; drugs used in combination or coincidental with the specific agent; and like factors well known in the medical arts.

In some embodiments, a pharmaceutical composition comprising a plantazolicin compound is administered once daily. In other embodiments, plantazolicin is administered twice daily, and in still other embodiments, it is administered three times a day. In some embodiments, a pharmaceutical composition comprises a plantazolicin compound in an amount from about 100 μg to about 100 mg. In other embodiments, the pharmaceutical composition comprises a plantazolicin compound in an amount from about 1 mg to about 50 mg.

As noted above, plantazolicin is bactericidal against B. anthracis, and as such pharmaceutical compositions described herein can be used to treat anthrax. Anthrax disease is caused by the bacterium Bacillus anthracis, a gram-positive, sporulating bacillus. B. anthracis is a soil bacterium and is distributed worldwide.

The disease can take on one of four forms: (1) cutaneous, the most common, which results from contact with an infected animal or animal products; (2) inhalational, which is less common and a result of spore deposition in the lungs, (3) gastrointestinal, and (4) oropharyngeal (back of the throat), both of which are due to ingestion of infected meat.

The cutaneous disease constitutes the majority (up to 95%) of anthrax cases. Anthrax usually develops in cattle, horses, sheep, and goats, and as such, is a major veterinary concern. Animals can contract the spores while grazing. Humans can contract anthrax from inoculation of minor skin lesions with spores from infected animals, their hides, wool or other products, such as infected meat (Franz et al. (1997) J. Am. Med. Assoc. 278(5): 399-411). Anthrax in humans is rarer than in animals unless the spores are spread intentionally.

Anthrax disease occurs when spores enter the body, germinate to the bacillary form, and multiply. In cutaneous disease, spores gain entry through cuts, abrasions, or in some cases through certain species of biting flies. Germination is thought to take place in macrophages, and toxin release results in edema and tissue necrosis but little or no purulence, probably because of inhibitory effects of the toxins on leukocytes. Generally, cutaneous disease remains localized, although if untreated it may become systemic in up to 20% of cases, with dissemination via the lymphatics. In the gastrointestinal form, B. anthracis is ingested in spore-contaminated meat, and may invade anywhere in the gastrointestinal tract. Transport to mesenteric or other regional lymph nodes and replication occur, resulting in dissemination, bacteremia, and a high mortality rate. Symptoms include nausea, loss of appetite, vomiting, fever, abdominal pain, vomiting of blood and severe diarrhea. Death results in 25%-60% of cases.

In cases of inhalation of anthrax spores, after deposition in the lower respiratory tract, spores are phagocytized by tissue macrophages and transported to hilar and mediastinal lymph nodes. The spores germinate into vegetative bacilli, producing a necrotizing hemorrhagic mediastinitis (Franz et al., supra). Symptoms include fever, malaise and fatigue, which can easily be confused with the flu. The disease may progress to an abrupt onset of severe respiratory distress with dyspnea, stridor, diaphoresis and cyanosis. Death usually follows within 24 to 36 hours.

The average incubation period for anthrax is 1 to 7 days, but it can take 60 days or longer for symptoms to develop. Symptoms depend on how the infection was acquired. For humans, anthrax is a particularly fearsome biological warfare agent, not only because of its deadliness, but also because anthrax weapons are relatively easy to produce and deliver. Production of anthrax spores requires little more than basic laboratory equipment and growth media. Anthrax weapons may be comprised of an anthrax source and an industrial sprayer that can produce aerosol particles of the appropriate size for victims to inhale. Such sprayers, for instance, can be mounted on low flying airplanes or other vehicles and used to spread anthrax over a wide area. Because of the ease and relatively small expense involved in producing and delivering anthrax weapons, such weapons are potentially highly attractive weapons of mass destruction for terrorist groups. Thus, in addition to potential organized military conflicts that may give rise to the use of such weapons, terrorist organizations are a potential threat for the use of such weapons in airports, office buildings and other centers of human activity.

Currently, B. anthracis infections are treated with various broad-spectrum antibiotics. In order to completely eliminate B. anthracis, antibiotic treatment often requires over 60 days of administration. Consequently, the current method of treatment increases the dangers of multi-drug resistance. Multi-drug resistance arises from horizontal gene transfer of drug-resistant bacteria and has lead to the generation of many harmful infectious diseases including, but not limited to, Vancomycin-resistant enterococcus (VRE) and Methicillin-resistance Staphlococcus aureus (MRSA).

Most current treatments of bacterial infections kill off the human intestinal bacteria which has two negative side effects: the “healthy” bacteria serve as a reservoir for antibiotic resistance and keep other pathogens at bay. Prolonged, broad-spectrum antibiotics leave patients at risk for secondary infections that are harder to treat that the primary infection. A plantazolicin-like compound and plantazolicin, both being highly discriminating antibiotics, provides numerous advantages over the currently used antibiotics, such as high specificity and low risk of developing multi-drug resistance.

Accordingly, it is one embodiment of the present invention to provide a method for treating or preventing anthrax in a patient by administering to the patient any of the pharmaceutical compositions comprising a plantazolicin-like compound or plantazolicin that are described herein. In one embodiment, the patient is a human. In another embodiment, the patient is an animal. The animal is preferably selected from a dog, cat, horse, sheep, goat, or cow. Any of the anthrax infections such as cutaneous, inhalation and gastrointestinal anthrax can be treated using a plantazolicin-like compound or plantazolicin. In some embodiments, a plantazolicin-like compound or plantazolicin is used to treat or prevent anthrax is administered orally, intravenously, subcutaneously or intramuscularly. In other embodiments, the daily dosage used to treat anthrax is from about 100 μg to about 100 mg of plantazolicin or plantazolicin-like compound, and in still other embodiments, the daily dosage of PZN or PZN-like compound is from about 1 mg to about 50 mg.

As noted above, B. pumilus produced PZN identical to the one produced by Bacillus amyloliquefaciens FZB42. The search for other PZN-like proteins was performed using a protein BLAST search where each PZN gene product was used as the query sequence. B. pumilus ATCC 7061 was a top result in the sequence search after FZB42, and the additional three PZN-like biosynthetic clusters were found in the Actinobacteria phylum including Clavibacter michiganensis subsp. sepedonicus (potato pathogen), Corynebacterium urealyticum DSM 7109 (human skin-associated bacterium, causative agent of some urinary tract infections), and Brevibacterium linens BL2 (human skin-associated bacterium).

Another embodiment of the present invention is to provide a method for identifying a plantazolicin-like protein, wherein the method comprises identifying a bacterial amino acid sequence exhibiting at least 50% amino acid identity to a plantazolicin precursor peptide from Bacillus amyloliquefaciens FZB42; obtaining a post-translationally modified product of the bacterial amino acid sequence; and testing the post-translationally modified product of the bacterial amino acid sequence in a Bacillus anthracis growth inhibitory assay, wherein the ability to inhibit the growth of Bacillus anthracis indicates that the bacterial amino acid encodes a plantazolicin-like protein.

The identification of a bacterial amino acid sequence exhibiting at least 50% amino acid identity to the plantazolicin precursor peptide can be used to search for a plantazolicin-like biosynthetic gene cluster in a bacterial genome. Alternatively, any other polypeptide from Bacillus amyloliquefaciens FZB42 can be used as a reference sequence to find other plantazolicin-like gene cluster products. By way of example and not of limitation, PznE can be used to search for other plantazolicin-like leader peptidases, which can then be used to search for plantazolicin-like biosynthetic clusters.

For sequence comparison, typically one sequence, such as plantazolicin in this case acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc. Acids Res. 12:387-395).

Another example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The protein sequence search shows sequences in order of highest to lowest sequence identity to the reference sequence. Any bacterial sequences identified in the protein sequence search as exhibiting at least 50% identity to the reference sequence can be further tested to confirm whether they are indeed plantazolicin-like sequences. One way of confirming is to test a post-translationally modified product of the bacterial amino acid sequence in a Bacillus anthracis growth inhibitory assay. In such an assay, the ability of the post-translationally modified product of the bacterial amino acid sequence to inhibit the growth of Bacillus anthracis indicates that the bacterial amino acid encodes a plantazolicin-like protein.

In an exemplary embodiment, the post-translationally modified product of the bacterial amino acid sequence can be obtained by growing bacteria containing a gene for the bacterial amino acid sequence under conditions, which allow for transcription of such gene, and for post-translational processing of a polypeptide encoded by the gene. One skilled in the art can determine such conditions without undue experimentation. Some of the parameters that can be varied include media compositions, aeration conditions, incubation times and temperatures.

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-interscience; Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; and the like. Relevant portions (or possibly the entirety) of each of these publications are herein incorporated by reference.

Having described the invention in detail, it will be apparent that modifications and variations are possible without departing from the scope of the invention defined in the appended claims.

### EXAMPLES

The following non-limiting examples are provided to further illustrate the present invention.

### Example 1

Strain Construction.

The B. amyloliquefaciens strains and plasmids used in this study are summarized in Table 1.

TABLE 1
Bacterial strains and plasmids used in this study

| | | |
|---|---|---|
| Strains | Description | Source/reference |

Bacillus subtilis

| | | |
|---|---|---|
| DSM10^{T} | 168, trpC2, type strain | DSMZ Braunschweig |
| CU1065 | 168, trpC2 attSPβ | Butcher et al., Mol |
| HB0042 | 168, trpC2 attSPβ sigW::kan | Microbiol. 60: 765-82, |
| | | 2006 |

Bacillus megaterium

| | | |
|---|---|---|
| 7A/1 | Indicator strain for polyketides | Laboratory stock |

Bacillus amyloliquefaciens

| | | |
|---|---|---|
| FZB42 | Wild type | Idriss et al., 2002, |
| | | Microbiology |
| | | 148: 2097-109 |
| CH5 | FZB42 sfp::ermAM yczE::cm | Chen, X. 2009. Whole |
| | | genome analysis of the |
| | | plant growth-promoting |
| | | Rhizobacteria Bacillus |
| | | amyloliquefaciens |
| | | FZB42 with focus on |
| | | its secondary |
| | | metabolites. |
| | | Dissertation. HU- |
| | | Berlin. |
| RSpMarA2 | Insertion of pMarA in CH5: degU::kan | Described herein |
| RS6 | sfp::ermAM, bac::cmR, deficient in lipopeptides, | Chen et al., 2009, J |
| | polyketides and bacilysin | Biotechnol. 140: 38-44 |
| RS26 (ΔpznB) | RS6 ΔRBAM_007480::spc does not produce | Described herein |
| | PZN | |
| RS27 (ΔpznI) | RS6 ΔRBAM_007440::spc produces PZN | Described herein |
| RS28 (ΔpznJ) | RS6 ΔRBAM_007450::spc does not produce | Described herein |
| | PZN | |
| RS29 (ΔpznF) | RS6 ΔRBAM_007400::spc produces PZN | Described herein |
| RS31 (ΔpznC) | RS6 ΔRBAM_007460::spc does not produce | Described herein |
| | PZN | |
| RS32 (ΔpznA) | RS6 ΔpznA::spc does not produce PZN | Described herein |
| RS33 (ΔpznL) | RS6 ΔRBAM_007500::spc produces desmethyl- | Described herein |
| | PZN, 1308 Da | |

Plasmids

| | | |
|---|---|---|
| pGEM-T | Ap^{r}, lacZ\grave{ } | Promega |
| pMarA | plasmid containing mariner transposon TnYLB-1 | Le Breton et al., 2006, |
| | | Appl Environ |
| | | Microbiol 72: 327-33 |
| pIC333 | plasmid with spc cassette | T. Msadek, Institute |
| | | Pasteur, Paris, France |
| pRS26a | pGEM-T with 2700 bp pznB | Described herein |
| pRS26b | pGEM-T with pznB::spc | Described herein |
| pRS27 | pGEM-T with SOE fusion-product | Described herein |
| | RBAM_007440/spc | |
| pRS28 | pGEM-T with SOE fusion-product | Described herein |
| | RBAM_007450/spc | |
| pRS29 | pGEM-T with SOE fusion-product | Described herein |
| | RBAM_007400/spc | |
| pRS31a | pGEM-T with 2600 bp pznC | Described herein |
| pRS31b | pGEM-T with pznC::spc | Described herein |
| pRS32a | pGEM-T with 2300 bp flanking region pznA | Described herein |
| pRS32b | pGEM-T with pznA::spc | Described herein |

Bacillus and indicator strains were cultivated routinely on Luria-Bertani broth (LB) medium solidified with 1.5% agar. For production of PZN, a medium containing: 40 g soy peptone, 40 g dextrin 10, 1.8 g KH₂PO₄, 4.5 g K₂HPO₄, 0.3 g MgSO₄×7H₂O, and 0.2 ml KellyT trace metal solution per L was used. KellyT trace metal solution: 25 mg EDTA disodium salt dihydrate, 0.5 g ZnSO₄×7H₂O, 3.67 g CaCl₂×2H₂O, 1.25 g MnCl₂×4H₂O, 0.25 g CoCl₂×6H₂O, 0.25 g ammonium molybdate, 2.5 g FeSO₄×7H₂O, 0.1 g CuSO₄×5H₂O; adjust to pH 6 with NaOH, 500 ml H₂O.

The media and buffers used for DNA transformation of Bacillus cells were prepared according to Kunst and Rapoport (J. Bacteriol. 177:2403-2407, 1995). Competent cells were prepared as previously described (Koumoutsi et al., 2004. J. Bacteriol. 186:1084-96). Mutants were obtained after transformation of the FZB42 derivatives with linearized, integrative plasmids containing resistance cassettes flanked by DNA regions homologous to the FZB42 chromosome. The oligonucleotides used for strain construction are listed in Table 2.

TABLE 2
Oligonucleotides used for gene replacement and Slice Overlap Extension
(SOE) PCR

| | |
|---|---|
| Oligonucleotide | Sequence (5′ to 3′) |

Spectinomycin resistance cassette

| | |
|---|---|
| Spc-fw | CTCAGTGGAACGAAAACTCACG (SEQ ID NO: 3) |
| Spc-rv | TAAGGTGGATACACATCTTGTC (SEQ ID NO: 4) |

pRS26a/b

| | |
|---|---|
| pznB-fw | ATCCATATCGCCAATCATACGG (SEQ ID NO: 5) |
| pznB-rv | GGAATCAATACCTGTCAGTTCG (SEQ ID NO: 6) |

pRS31a/b

| | |
|---|---|
| pznD-fw | ATTGACTAGGAGGTATTGGACG (SEQ ID NO: 7) |
| pznD-rv | TTCTATTGAATAGGAGGAGGCG (SEQ ID NO: 8) |

pRS32a/b

| | |
|---|---|
| 007400cst-fw | TGGAATGCTCTTTCCGCAGTAC (SEQ ID NO: 9) |
| 007400cst-rv | GTAACTCTGTTTCCACGTAACC (SEQ ID NO: 10) |

SOE PCR

| | |
|---|---|
| 7400 rv | TCTTCATCACGCAAATCAGTGC (SEQ ID NO: 11) |
| 7400 fw | CCGCATAAACGGGAATTGGAAG (SEQ ID NO: 12) |
| Spc in 7410 | TCTATAGAAACTTCTCTCAATTAGAAAAGAAAAGGGCAAGGAAA |
| | TGAG (SEQ ID NO: 13) |
| 7410 in spc | ACTCATTTCCTTGCCCTTTTCTTTTCTAATTGAGAGAAGTTTCTAT |
| | AG (SEQ ID NO: 14) |
| Start in spc | CTTTGTAAAAAGAGGAGCCTGTCTTATGAGCAATTTGATTAACGG |
| | (SEQ ID NO: 15) |
| Spc in start | TTTTTCCGTTAATCAAATTGCTCATAAGACAGGCTCCTCTTTTTAC |
| | AAAG (SEQ ID NO: 16) |
| 7430 in spc | GCTGGGACTAAAAGGAGAGCGGGAAATGAGCAATTTGATTAACG |
| | G (SEQ ID NO: 17) |
| Spc in ORF2 | TTCTATAGAAACTTCTCTCAATTAGATTTAATATAAAGAAGCATA |
| | GACC (SEQ ID NO: 18) |
| Spc in 7430 | TTTTTCCGTTAATCAAATTGCTCATTTCCCGCTCTCCTTTTAGTCC |
| | CAGC (SEQ ID NO: 19) |
| ORF2 in spc | TGGTCTATGCTTCTTTATATTAAATCTAATTGAGAGAAGTTTCTAT |
| | AG (SEQ ID NO: 20) |
| 7440 rv | TCACGTCCAATACCTCCTAGTC (SEQ ID NO: 21) |
| 7440 fw | ATCGACAGAGGGCAGATTATCG (SEQ ID NO: 22) |
| ORF2 in spc for 7450 | GATTATTGACTAGGAGGTATTGGACATGAGCAATTTGATTAACG |
| fw | G (SEQ ID NO: 23) |
| 7460 in spc for 7450 | GTTTGTTGAGACATCTGTATTCCTCCCTAATTGAGAGAAGTTTCT |
| rv | ATAG (SEQ ID NO: 24) |
| 7450 rv | TAATGTCGTCCATTTACTCACC (SEQ ID NO: 25) |
| 7450 fw | TTGGCTCGAATAAATGTTGACC (SEQ ID NO: 26) |
| Spc in ORF2 for 7450 | TTTTTCCGTTAATCAAATTGCTCATGTCCAATACCTCCTAGTCAAT |
| rv | AATC (SEQ ID NO: 27) |
| Spc in 7460 for 7450 | TTCTATAGAAACTTCTCTCAATTAGGGAGGAATACAGATGTCTCA |
| fw | ACAAAC (SEQ ID NO: 28) |
| End in spc for 7500 | CGCTTAGACCCTAAAGATATACTTTCTCTAATTGAGAGAAGTTTC |
| rv | TATAG (SEQ ID NO: 29) |
| 7490 in spc for 7500 | AACTCTTTGGAGGTGTCACAGTTATATGAGCAATTTGATTAACGG |
| fw | (SEQ ID NO: 30) |
| 7500 fw | AAGGTCCTAGACGCCCTATTCC (SEQ ID NO: 31) |
| 7500 rv | GATGTGTAGTTTTCAACGCTCG (SEQ ID NO: 32) |
| Spc in end for 7500 | CTATAGAAACTTCTCTCAATTAGAGAAAGTATATCTTTAGGGTCT |
| fw | AAGCG (SEQ ID NO: 33) |
| Spc in 7490 for 7500 | CCGTTAATCAAATTGCTCATATAACTGTGACACCTCCAAAGAGTT |
| rv | TACC (SEQ ID NO: 34) |

Primers for RT-PCR

| | |
|---|---|
| pznF (fw and rv) | GGATTATTGCGTACTCCGTTTC (SEQ ID NO: 35) |
| | CTGCCTCCGCCAATAAATG (SEQ ID NO: 36) |
| pznK (fw and rv) | ATGCCAAAGTACGGTTGGG (SEQ ID NO: 37) |
| | CTCCTTGTAGGCTGCTTTCC (SEQ ID NO: 38) |
| pznG (fw and rv) | CCACAGGATATCAGCCTTGAAG (SEQ ID NO: 39) |
| | CGATAATCTGCCCTCTGTCG (SEQ ID NO: 40) |
| pznH (fw and rv) | CGCTCGCTCAAATTGAAACG (SEQ ID NO: 41) |
| | ACAACAACCCACAGATACGC (SEQ ID NO: 42) |
| pznI (fw and rv) | TAGCCTGGAAGCAGAGGGTA (SEQ ID NO: 43) |
| | ACTTTTGGCAGGTGACAACC (SEQ ID NO: 44) |
| pznA (fw and rv) | GGAGGAGGTAACAATTATGACTCAA (SEQ ID NO: 45) |
| | GGTACAGGTACAGCGTGCAG (SEQ ID NO: 46) |
| pznJ (fw and rv) | TTGGATATCGGAATCGAGTTG (SEQ ID NO: 47) |
| | CGGATGCCCAATTATCTGTT (SEQ ID NO: 48) |
| pznC (fw and rv) | TCATGTCCCTTGGTTGTGTG (SEQ ID NO: 49) |
| | GCCGTGATACCATACTTGAGG (SEQ ID NO: 50) |
| pznD (fw and rv) | CGCGATGTAGATGACGTTTG (SEQ ID NO: 51) |
| | GATTGGCGATATGGATTAGTTG (SEQ ID NO: 52) |
| pznB (fw and rv) | AAGGCATGCCACTAATTTGG (SEQ ID NO: 53) |
| | GATAAAGAGCTCCGCCAGAA (SEQ ID NO: 54) |
| pznE (fw and rv) | CATAGCAATAATGCGTACGGTG (SEQ ID NO: 55) |
| | GAGACATTGTCGGCGAAGA (SEQ ID NO: 56) |
| pznL (fw and rv) | GATGAGAGGGAAACCTCATCC (SEQ ID NO: 57) |
| | CTCCCAAACTGTTCCTGTCC (SEQ ID NO: 58) |

Spectinomycin (90 μg/ml) was used for selecting transformants. Gene interruption strains were obtained as follows: PznB RS26: A 2.7 kb PCR fragment was amplified from FZB42 chromosomal DNA using primers pznB-fw and pznB-rv. The fragment was then cloned into pGEM-T, yielding plasmid pRS26a. Plasmid pRS26b was obtained by insertion of a spectinomycin resistance cassette, which was subcloned by PCR using the spc-fw and spc-rv primers and the pIC333 plasmid as a template. The cassette was placed into the central region of the insert and digested with BglII and BamHI. PznC RS31: With primers pznC-fw and pznC-rv, a 2.6 kb fragment containing pznC was amplified by PCR and cloned into vector pGEM-T-Easy yielding plasmid pRS31a. A central fragment of the insert was removed by digestion with Eco105I and replaced with the spectinomycin resistance cassette, yielding pRS31b. PznA RS32: With primers 007400cst-fw and 007400cst-rv, a 2.3 kb fragment encoding the unannotated precursor peptide, pznA, was amplified by PCR and cloned into vector pGEM-T-Easy, yielding plasmid pRS32a. The precursor peptide gene was cleaved by Bsp14071 and interrupted by insertion of a spectinomycin resistance cassette, yielding pRS32b.

The mutants RS27, RS28, RS29 and RS33 were generated by gene splicing using the overlapping extension (SOE) method (Horton et al, 1990, Biotechniques 8:528-35). This method assists in avoiding possible polar effects caused by interrupted reading frames. SOE PCR fusion products were generated using the primers listed in Table 2 and the spectinomycin gene of pIC333. A-tailing of the Pfu-PCR product was performed according to the Promega pGEM-T protocol and ligated into pGEM-T yielding pRS27, pRS28 and pRS29. For mutant RS33, the PCR product was used directly for transformation.

Mutant RSpMarA2 was isolated from a mariner-based (pMarA) transposon library prepared in strain CH5 according to Breton et al. (Appl Environ Microbiol 72:327-33, 2006). In this transposon mutant, pMarA was integrated into the degU gene, which is a global transcriptional regulator that activates the bacillomycin D promoter.

Bioassay.

LB-Agar (20 ml) was mixed with 0.5 ml of the indicator strain (OD₆₀₀˜1.0). 10 μl of purified PZN suspended in water was spotted on the agar and incubated for 16 h at 22° C. The growth suppression activity of PZN was observed as clear zone.

Purification of Plantazolicin.

A cell surface extract from a 250 ml culture of strain RSpMarA2 was collected using the previous method. During concentration under reduced pressure, plantazolicin precipitated. The precipitate was washed 3 times with deionized water, resulting in crude, desalted plantazolicin. Pure plantazolicin was obtained using RP-HPLC (Grom-Sil ODS-5 ST, 20×250 mm, Alltech-Grom, Rottenburg-Hailfingen) with a linear gradient elution of 40-70% aqueous acetonitrile with 0.1% v/v formic acid over 40 min at a flow rate of 15 ml/min.

RT-PCR.

Total RNA was isolated with the Qiagen RNeasy Mini Kit. Cells (1.0 OD₆₀₀) were harvested from M9 minimal media supplemented with BME vitamin mix (Cat. No. B6891) and ATCC trace mineral solution (Cat. No. MD-TMS) and treated with the Qiagen RNAprotect Bacteria Reagent. Harvested cells were resuspended in 250 μl of 10 mM tris (pH 8.5) with 15 mg/ml lysozyme and 5 μl proteinase K (20 mg/ml) and digested for 1 h at 22° C. with gentle agitation. A DNase I digestion was performed for pznE and pznL using the Qiagen RNase Free DNase set. DNase I (7 μl) and RDD DNA digest buffer (7 μl) were used to hydrolyze contaminating DNA for 20 min at 22° C. The RNA isolation protocol was then followed to manufacturer's instructions. To minimize background, a DNase I digestion (5 μl) was executed to the RNA samples and placed at 37° C. for 20 min. It should be noted that this was the second DNase I digest for pznE and pznL. Samples were column purified using the RNA cleanup protocol in the RNeasy Mini Handbook (Qiagen). Digestion and cleanup were repeated for all RNA samples, excluding those used to analyze pznE and pznF. cDNA was prepared with commercially available RT-PCR kits using 1 μg of RNA and the primers listed in Table 2.

Reverse Transcriptase-PCR.

Transcription of all 12 pzn genes in M9 minimal media was confirmed by RT-PCR. All amplicons migrated with their expected sizes (Table 2). In addition to confirming transcription, the intergenic regions of the PZN biosynthetic cluster were assessed to determine if the mRNA was polycistronic. Using the appropriate primers from adjacent genes, it was determined that the biosynthetic genes were transcribed into two polycistronic mRNAs (pznFKGHI and pznJCDBEL) and a monocistronic mRNA for pznA. Amplification of the region between pznE and pznL resulted in a band that was visible only under extreme contrast (data not shown).

Plantazolicin was tested for its ability to inhibit the growth of Gram-positive bacteria. In the agar bioassays, where growth inhibition is indicated by a clear zone, plantazolicin was shown to be growth inhibitory towards most of the gram-positive Bacilli surveyed, especially B. megaterium and B. subtilis HB0042 (Table 3).

TABLE 3
Activity spectrum of plantazolicin

| Indicator strain | Result^{a} | Source or reference^{b} |
|---|---|---|
| Bacillus brevis ATCC8246 | + | ATCC |
| Bacillus subtilis 168 | + | Peric-Concha and |
| | | Long (2003) Drug |
| | | Discov Today 8, |
| | | 1078-1084 |
| Bacillus cereus ATCC14579 | + | ATCC |
| Bacillus licheniformis ATCC9789 | + | ATCC |
| Micrococcus luteus | + | Laboratory collection |
| Bacillus pumilus | − | Laboratory collection |
| Bacillus subtilis CU1065 | + | Peric-Concha and |
| | | Long (2003) Drug |
| | | Discov Today 8, |
| | | 1078-1084 |
| Bacillus subtilis HB0042 | ++ | Peric-Concha and |
| | | Long (2003) Drug |
| | | Discov Today 8, |
| | | 1078-1084 |
| Bacillus sphaericus | + | Laboratory collection |
| Paenibacillus polymyxa | − | Laboratory collection |
| Paenibacillus granivorans | + | Laboratory collection |
| Bacillus megaterium 7A1 | ++ | Laboratory collection |
| Arthrobacter sp. | − | Laboratory collection |
| Staphylococcus aureus | − | Laboratory collection |
| E. coli K12 | − | Laboratory collection |
| Klebsiella terrigena | − | Laboratory collection |
| Pseudomonas sp. | − | Laboratory collection |
| Erwinia caratovora | − | Laboratory collection |

^{a}Degree of inhibition in a bioassay: ++: inhibition; +: weak inhibition; −: no inhibition
^{b}ATCC: American Type Culture Collection

### Example 2

Production and Purification of PZN.

Overnight cultures (4×20 mL) of B. amyloliquefaciens FZB42 strain RSpMarA2 (Δsfp, yczE, degU) were used to inoculate 4×6 L flasks with 2 L of Luria Burtani (LB) broth supplemented with chloramphenicol (7 μg/mL) and kanamycin (7 μg/mL). Cultures were grown with shaking for 48 h at 37° C. Cells were harvested by centrifugation (4,000×g), washed with Tris-buffered saline (pH 8.0), and harvested a second time. Crude PZN was obtained by a non-lytic, methanolic extraction of the cellular surface. Cells were resuspended in MeOH (10% culture volume) and anhydrous Na₂SO₄ (5 g/L culture). The cell mixture was agitated by vortex (45 s) and equilibrated for 15 min at 22° C. The supernatant was retained after centrifugation (4,000×g), vacuum filtered with Whatman filter paper, and rotary evaporated to dryness to yield about 100 mg/L of a yellowish-brown solid. This crude material was dissolved in 80% aqueous MeCN (10 mL for 8 L culture), where the sample separated into two layers. The top organic layer was retained and concentrated for injection onto an Agilent 1200 series liquid chromatograph that was fitted inline to an Agilent 6100 Series Quadrupole LC/MS. For preparative purposes, PZN was reverse phase purified using a Thermo BETASIL C18 column (250 mm×10 mm; pore size: 100 Å; particle size: 5 μm) at a flow rate of 4 mL/min. A gradient of 65-85% MeOH with 0.1% formic acid over 32 min was used. The fractions containing PZN (as monitored by A₂₆₆ and MS) were collected into 20 mL borosilicate vials and the solvent removed in vacuo. The isolated yield for PZN following this procedure was routinely 150-200 μg/L culture. Mutant RS33 (Asfp, bac, pznL) was prepared similarly, with the only exceptions being a 24 h fermentation, substitution of spectinomycin (90 μg/mL) for kanamycin, and elimination of the TBS wash.

Production of PZN (Elevated Oxygen).

Increased aeration of B. amyloliquefaciens FZB42 strains RSpMarA2 and RS33 was achieved using a New Brunswick Scientific BioFlo 110 Fermenter system. RSpMarA2 and RS33 (9 L) were cultured at 37° C. with 250 rpm stirring for 24 h. Air was supplied at 5 L/min (saturated in oxygen, ˜1 L/min).

Determination of Minimum Inhibitory Concentration (MIC).

B. anthracis strain Sterne was grown to stationary phase in a 10 mL LB culture at 37° C. The culture was adjusted to OD₆₀₀ of 0.01 in LB broth and added to 96-well plates. 2-fold dilutions of PZN (5 mg/mL in 80% MeCN) were added to the cultures (0.5-128 μg/mL). Kanamycin was added similarly to control samples, with dilutions from 1-32 μg/mL. Covered plates were incubated at 37° C. for 12 h. The minimum inhibitory concentration that suppressed the growth of at least 99% of the bacteria (MIC₉₉) was established based on culture turbidity. Additional pathogens were grown and prepared similarly as above, with the exception of optimizing the growth media to match an organism's nutritional requirements (Streptococcus pyogenes, Todd Hewitt broth; Listeria monocytogenes, Enterococcus faecalis st. U503 [VRE], and Staphylococcus aureus st. NRS384/USA300 [MRSA], brain heart infusion). Positive controls: S. pyogenes and L. monocytogenes, kanamycin; E. faecalis, tetracycline; S. aureus, vancomycin). Bactericidal activity was determined by diluting 1 μL of B. anthracis strain Sterne grown with 8 μg/mL PZN into 99 μL of media. The sample was then streaked onto LB agar plates and incubated for 24 h for counting colony-forming units.

Agar Diffusion Bioassay.

B. anthracis strain Sterne was grown as described previously and diluted to OD₆₀₀ of 0.13. The diluted culture (100 μL) was inoculated onto an LB plate and allowed to dry. HPLC-purified PZN (50-200 μg) was added to a paper disk, dried, and added to the plate. Cultures were then incubated at 37° C. for 12 h. Kanamycin (8-25 μg) was used as a positive control, and 80% MeCN was the negative (solvent) control.

Microscopy.

Differential interference contrast (DIC) microscopy images were obtained by preparing live cell images of B. anthracis cultures. Samples were pretreated with or without PZN at 4 μg/mL (MIC₉₉), and morphology was assessed using a Zeiss LSM 700 microscope. The objective used was a Plan-Apochromat 63×/1.40 Oil DIC M27. The analysis software used was Program Zen 2009 Light Edition.

On-Line RPLC-FTMS.

All reverse phase liquid chromatography (RPLC)-Fourier-transform mass spectrometry (FTMS) was conducted using an Agilent 1200 high performance LC(HPLC) system with an autosampler coupled directly to a ThermoFisher Scientific LTQ-FT hybrid linear ion trap-FTMS system operating at 11 tesla. The MS was calibrated weekly using the calibration mixture and instructions specified by the manufacturer. All instrument parameters were tuned according to the manufacturer's instructions (employing bovine ubiquitin for tuning purposes). For all analyses of PZN, a 1 mm×150 mm Jupiter C18 column (Phenomenex, 300 Å, 5 μm) was connected in-line with the electrospray ionization source (operated at ˜5 kV with a capillary temperature of 200-250° C.) for the MS system. A typical sample was loaded onto the column using the autosampler and separated using a linear gradient of H₂O/MeCN and 0.1% formic acid with the analytes eluted directly into the MS. All ionized species were subjected to an MS method with five MS and MS/MS events: 1) full scan measurement of all intact peptides (all ions detected in the FTMS in profile mode; resolution: 100,000; m/z range detected: 400-2000), 2-5) data-dependent MS/MS on the first, second, third, and fourth most abundant ions from scan (1) using collision induced dissociation (CID) (all ions detected in the FTMS in profile mode; minimum target signal counts: 5,000; resolution: 50,000; m/z range detected: dependent on target m/z, default charge state: 2, isolation width: 5 m/z, normalized collision energy (NCE): 35; activation q value: 0.40; activation time: 30 ms). During all analyses, dynamic exclusion was enabled with the following settings: repeat count—2, repeat duration—30 s, exclusion list size—300, exclusion duration—60 s.

Direct Infusion FTMS.

After lyophilization for at least 24 h, HPLC purified samples were dissolved in 80% MeOH (to ˜0.5 mg/mL) and then further diluted 10-fold into 50% MeOH supplemented with 0.1% formic acid. The diluted samples were directly infused using an Advion Nanomate 100. The singly charged ions were targeted for CID using identical settings as above, except that the resolution was set to 100,000.

N-Terminal Labeling.

Purified PZN and desmethylPZN were dissolved in 80% MeCN, 10 mM MOPS (pH 8.0) to a final concentration of 1.5 mM. An aliquot (5 μL) was transferred to a microfuge tube containing 5 μL of 80% MeCN, 10 mM MOPS (pH 8.0) supplemented with 20 mM EZ-Link® sulfo-NHS-biotin. Control reactions lacked the NHS-biotin reagent. The samples were allowed to react for 3 h at 23° C. prior to analysis on an Applied Biosystems Voyager DE-STR MALDI-TOF-MS.

NMR.

PZN was produced from low oxygenation cultures and purified as described above. PZN (700 μg) was dissolved in 200 μL of DMSO-d₆ and placed into an Advanced Shigemi 5 mm NMR tube matched to DMSO-d₆. NMR experiments were conducted on a Varian Unity Inova 500 NB (¹H-¹H-gCOSY) and a Varian Unity Inova 600 spectrometer (¹H, ¹H-¹H-TOCSY and ¹H-¹³C-gHMBC) using a 5 mm Varian ¹H{¹³C/¹⁵N} PFG Z probe and 5 mm Varian ¹H{¹³C/¹⁵N} XYZ PFG triple resonance probe, respectively. The ¹H-NMR, TOCSY and gHMBC experiments were conducted at 25° C. and utilized water suppression. A mixing time of 150 ms was used for the TOCSY. For the gHMBC, ¹J and ⁿJ were set to 140 and 8 Hz, respectively. Chemical shifts were referenced using DMSO (δ_{H}=2.50 and δ_{C}=39.51), and the spectra were processed and analyzed using MestReC. Stereochemical configuration was assumed to be identical to the ribosomally produced precursor peptide.

Production of PZN from Bacillus pumilus ATCC 7061.

Cultures were prepared as described above for Bacillus amyloliquefaciens FZB42 cells, with the exception that no antibiotics were added. The method employed for metabolite extraction and HPLC purification were identical to samples from B. amyloliquefaciens. Purified fractions were analyzed on a Bruker Daltonics ultrafleXtreme MALDI-TOF/TOF instrument operating in reflector/positive mode. Sinapic acid was used as the matrix.

Results.

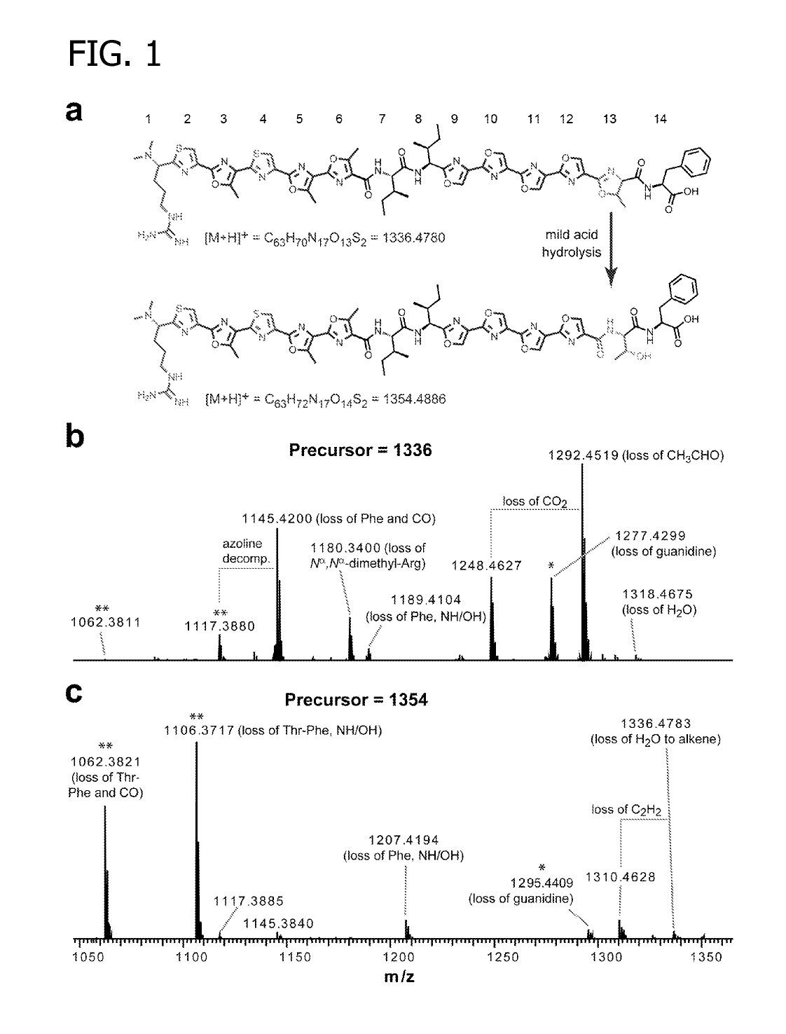

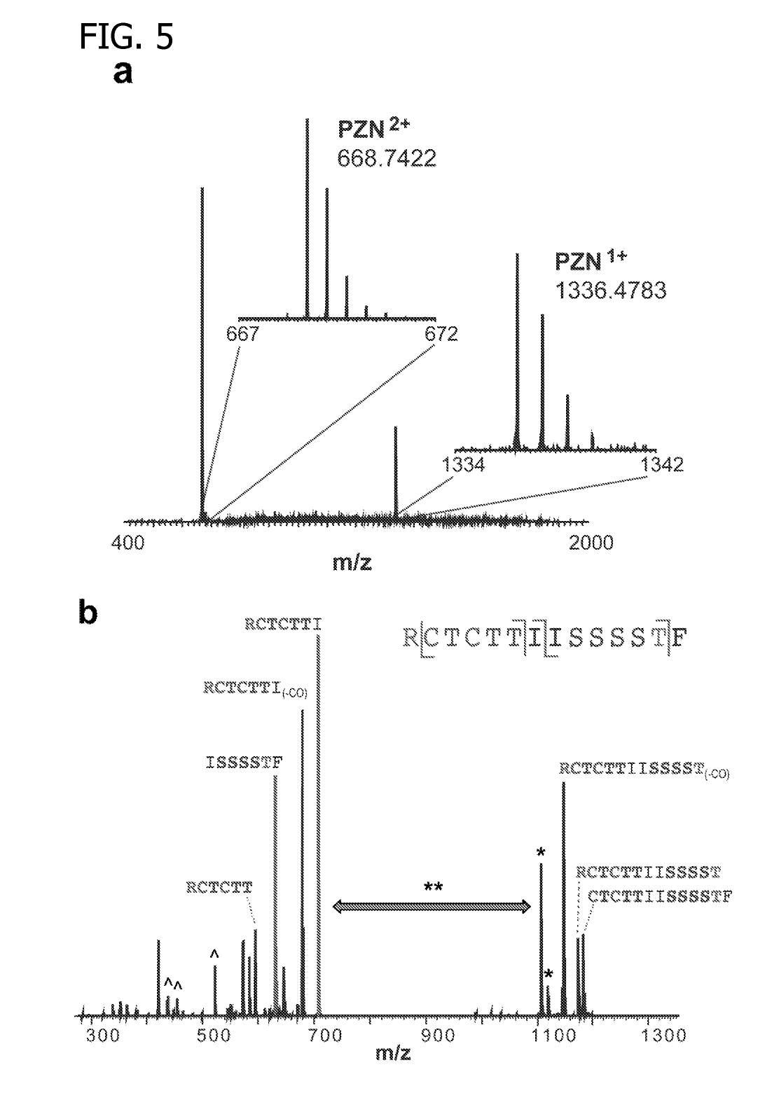

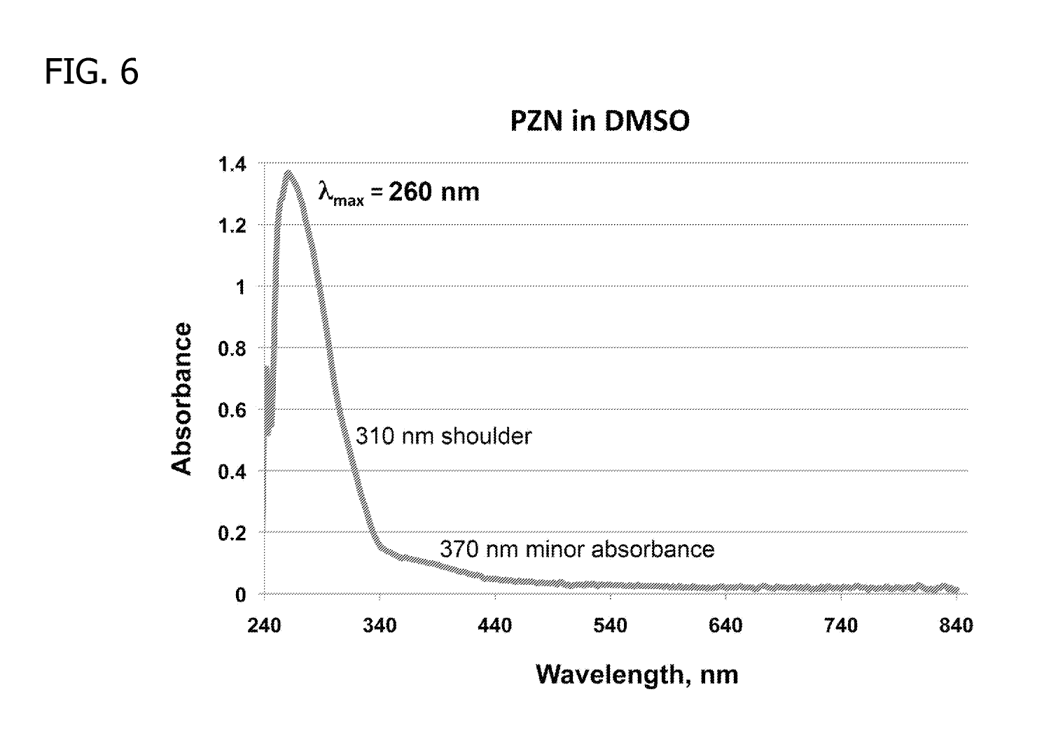

Mass spectrometry (MS) was used as the main spectroscopic tool in elucidating plantazolicin structure. Through the use of high-resolution, linear ion trap Fourier Transform hybrid MS (LTQ-FT) operating at 11 tesla, the mass of the protonated form of PZN was measured to be 1336.4783 Da (FIG. 1a and FIG. 5). Due to the high mass accuracy of FT-MS and the known sequence of the core region of the precursor peptide (₁RCTCTTIISSSSTF₁₄) (Lee et al., (2010) Proc Natl Acad Sci USA 105, 5879-5884, Scholz et al., 2011, J Bacteriol 193, 215-224), the molecular formula of [PZN+H]⁺ could be deduced (C₆₃H₇₀N₁₇O₁₃S₂; theoretical monoisotopic mass=1336.4780 Da; error, 0.15 ppm). This formula required that 9 out of 10 heterocyclizable residues (Cys, Ser, Thr) in the core region of the precursor peptide be converted to the azole heterocycle (FIG. 1A). Due to their adjacent positions, these processed residues form a contiguous polyazole, which was supported by spectrophotometric analysis. PZN gave absorption bands at 260 nm (λₘₐₓ), 310 nm (minor shoulder), and 370 nm (very weak shoulder), indicating the presence of a complex chromophore (FIG. 6). The remaining heterocyclizable residue was left at the azoline (thiazoline, oxazoline, or methyloxazoline) oxidation state. Also, this formula required leader peptide cleavage after Ala-Ala and two methylation events, consistent with earlier deletion studies (Scholz et al., supra).

Collision induced dissociation (CID) was then used to localize the site(s) of dimethylation and the azoline heterocycle. Analysis of the doubly charged PZN ion using in-line HPLC-FTMS resulted in a spectrum that was featureless from m/z ˜700-1100 as a result of contiguous heterocycle formation (FIG. 5). Nonetheless, the production of several diagnostic fragment ions was noted including peptide bond cleavage at Ile-Ile. The masses of these resultant ions demonstrated that the N-terminal (b⁺ ion) fragment contained both posttranslational methyl groups and that the C-terminal (y⁺ ion) fragment contained the azoline (which was now restricted to either oxazoline or methyloxazoline due to the absence of Cys on this fragment). Other informative fragment ions were derived from cleavage between Arg1-Cys2(thiazole) and Thr13(methyloxazoline)-Phe14. The former cleavage event demonstrated that both posttranslational methyl groups were localized to Arg1.

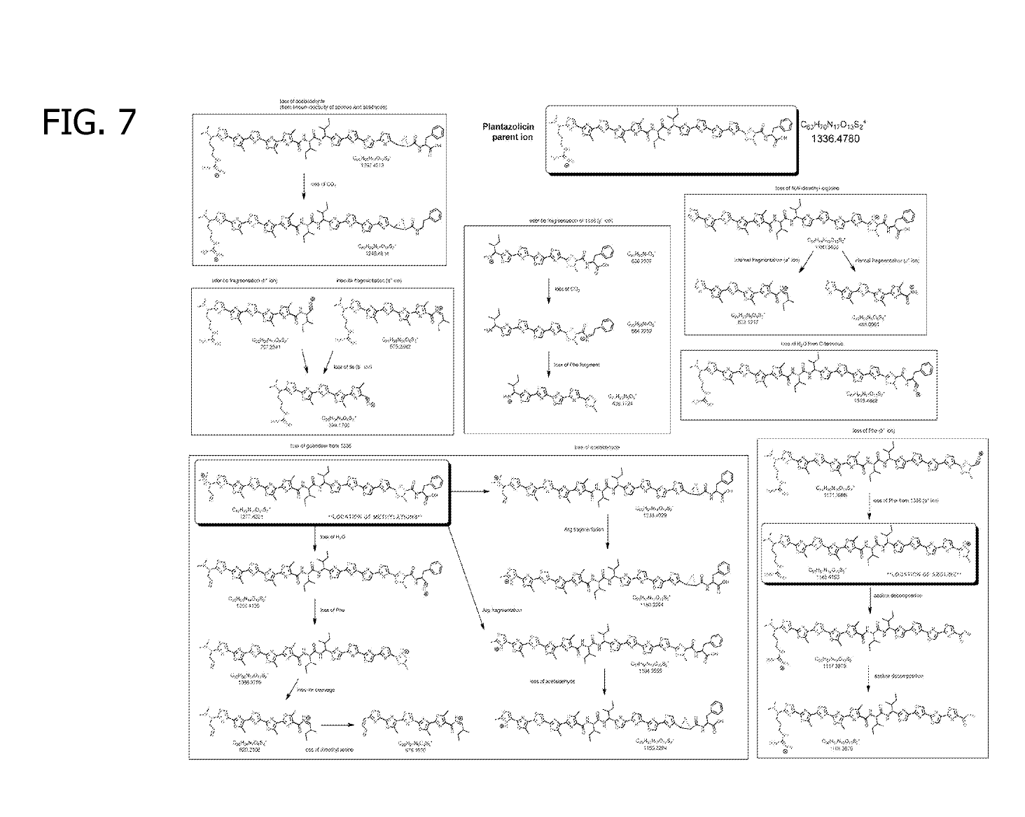

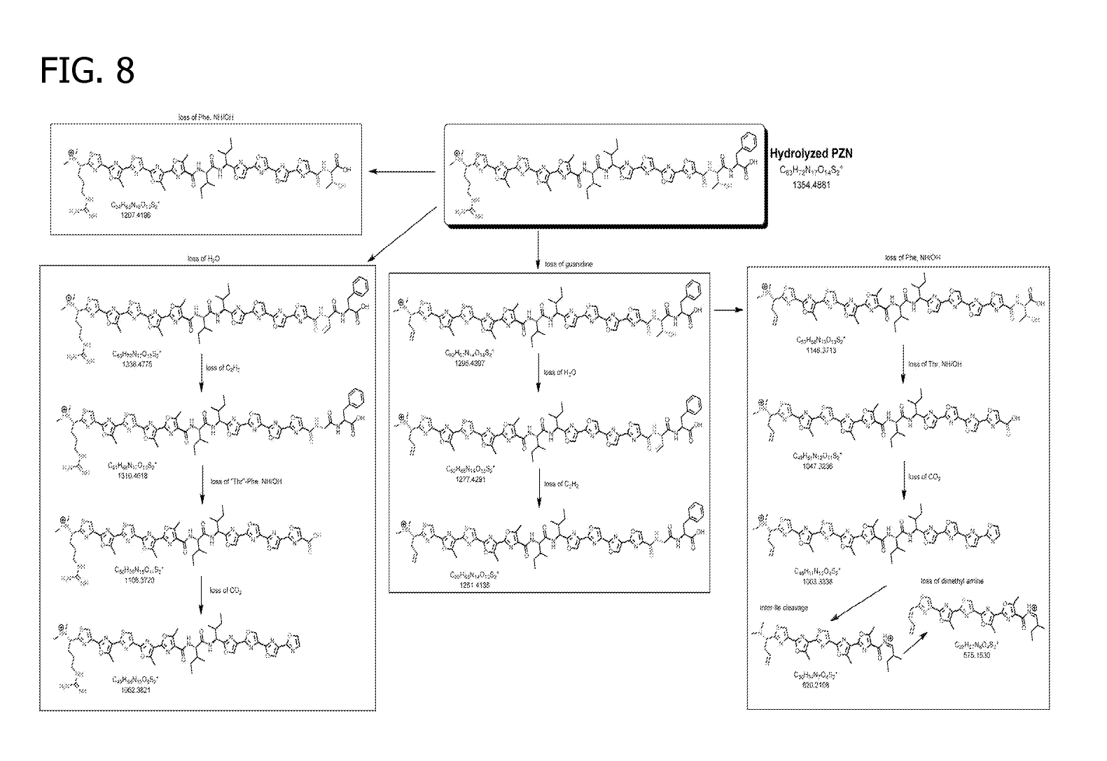

Cleavage between Thr13-Phe14 led to the formation of several decomposition products that permitted the localization of the (methyl)oxazoline to Thr13. From the apparently unstable parent ion, a formal loss of allene from methyloxazoline (C₃H₄, 40.0313 Da) to yield a C-terminal amide was frequently observed (FIG. 5). Further support for the location of the azoline heterocycle came from hydrolysis studies, as discussed below. Proposed structures for all assignable ions are given in FIGS. 7 and 8.

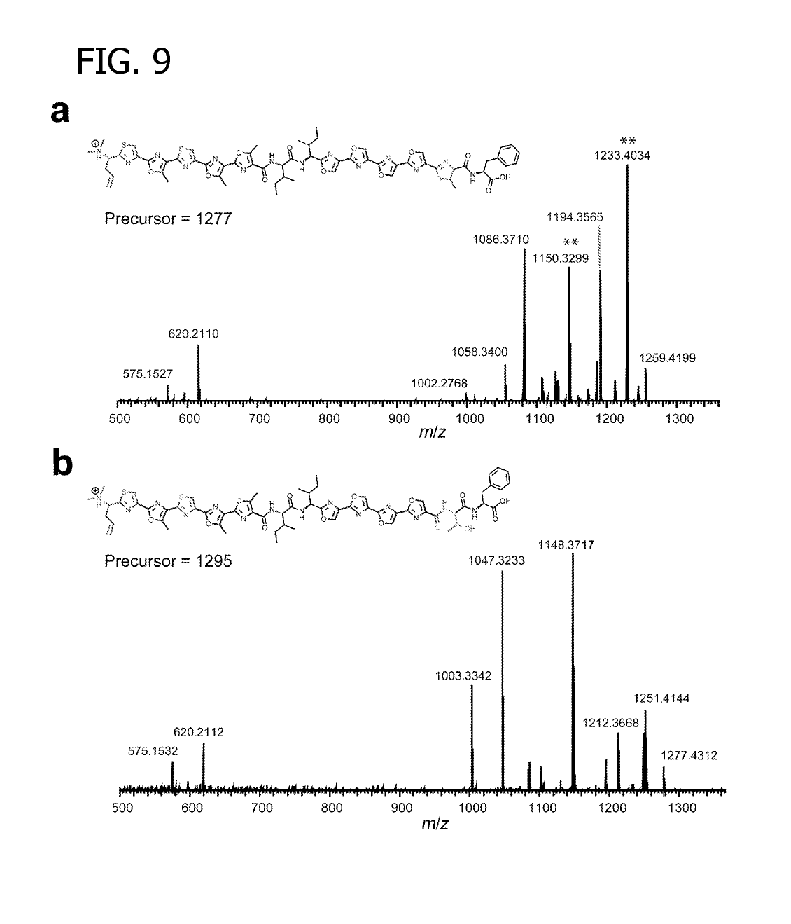

Upon in-depth FTMS analysis of singly charged PZN, which was introduced by direct infusion, much larger ions relative to doubly charged PZN parent ions were routinely observed including the ones consistent with the loss of guanidine (−59.0483 Da, m/z 1277.4299; error, 0.16 ppm) (FIG. 1B). This indicated that the site of dimethylation was restricted to either the N-terminal amine or the alkyl sidechain of Arg1. The latter was thought to be highly improbable since the enzyme known to catalyze this reaction (PznL) was predicted by sequence alignment to be a S-adenosylmethionine (SAM)-dependent methyltransferase. The only SAM-dependent enzymes capable of engaging in C—H bond activation are the radical SAM enzymes, which are identifiable by numerous conserved Cys that form Fe—S clusters, which are lacking in PznL (Leet et al., supra, Scholz et al., supra). Higher order CID was performed on the deguanidinated form of PZN (m/z 1277), providing corroborating evidence for N-terminal dimethylation (FIG. 9). In addition to CID analysis, further support for the N-terminus being the site of dimethylation in PZN came from chemoselective labeling.

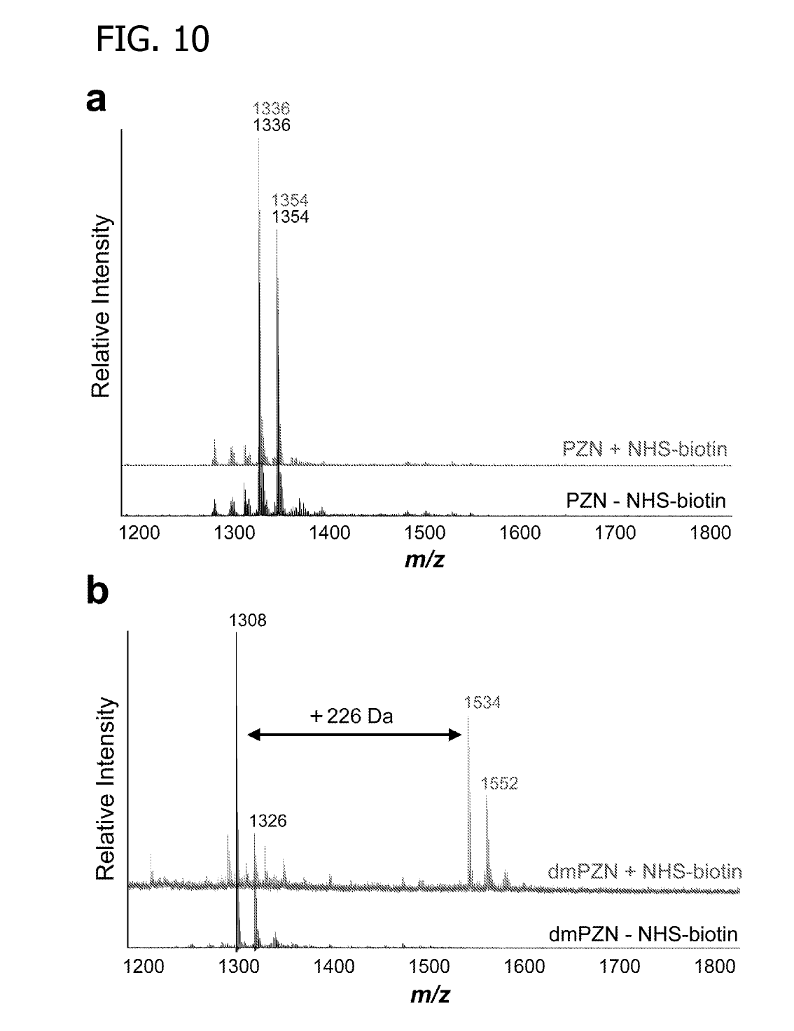

HPLC-purified PZN and desmethylPZN (from the pznL methyltransferase deletion strain) were reacted with the amine-specific reagent, N-hydroxysuccinimide (NHS)-biotin (Sholz et al., supra). As observed by MALDI-MS, labeling was only successful in the desmethylPZN reaction, indicating the presence of a free amine in this compound, but not in PZN (FIG. 10). From this, it has become clear that the leader peptide cleavage occurs before methylation, and that the ABC transport system does not distinguish between PZN and desmethylPZN.

From the apparent hydrolysis of PZN following SDS-PAGE, it was shown that PZN contained an azoline. Such heterocycles are prone to both acid- and base-catalyzed hydrolysis (Frump, J. A. (1971) Chemical Reviews 71, 483; Martin et al., Journal of the American Chemical Society 83, 4835-4838). Mild acid treatment of PZN yielded m/z 1354 (+18), which was shown by CID studies to be from the reconstitution of the Thr13 residue of the precursor peptide (FIG. 1B). Higher order tandem MS experiments further confirmed the location of the PZN methyloxazoline moiety (FIGS. 8 and 9). It is interesting to note that this methyloxazoline was the sole heterocycle not processed by the TOMM dehydrogenase, suggesting that the FMN-dependent dehydrogenase (B) is capable of distinguishing this heterocycle from others during the biosynthetic process. During the extensive MS analysis of PZN, it was noticed that fragmentation of the methyloxazoline moiety gave rise to a characteristic mass loss. CID fragmentation of PZN yielded an intense daughter ion of m/z 1292.4519 (FIG. 1B). The mass difference from the PZN parent ion was 44.0261 Da, which was consistent with the neutral loss of acetaldehyde (C₂H₄O, exact mass=44.0262). Loss of acetaldehyde is conceivable from cyclo-elimination of methyloxazoline to yield a nitrile ylide, which can re-cyclize to form an azirine. The microscopic reverse of this reaction pathway is well known in the chemical literature where azirines are reacted with aldehydes to form oxazolines via 1,3-dipolar cycloaddition (Frump, J. A. supra, Giezenda et al., 1973, Helvetica Chimica Acta 56, 2611-2627; Sa et al., 1996, Journal of Organic Chemistry 61, 3749-3752). Of note, the loss of acetaldehyde was observed only when methyloxazoline was present on the parent ion (see FIG. 1B-1C and FIGS. 7-9).

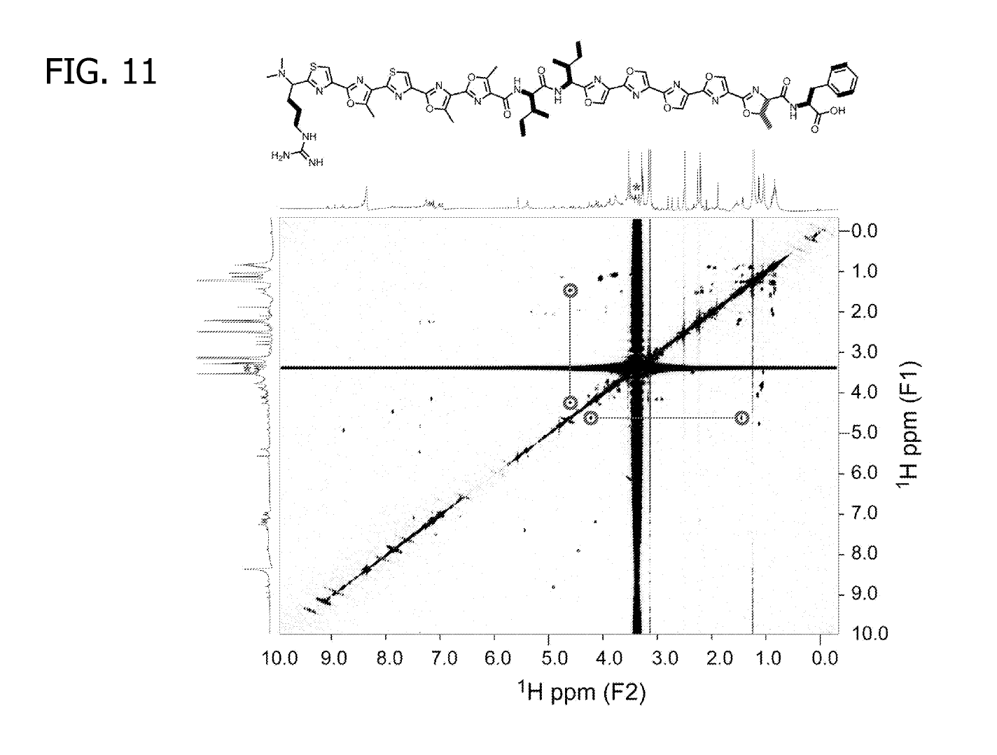

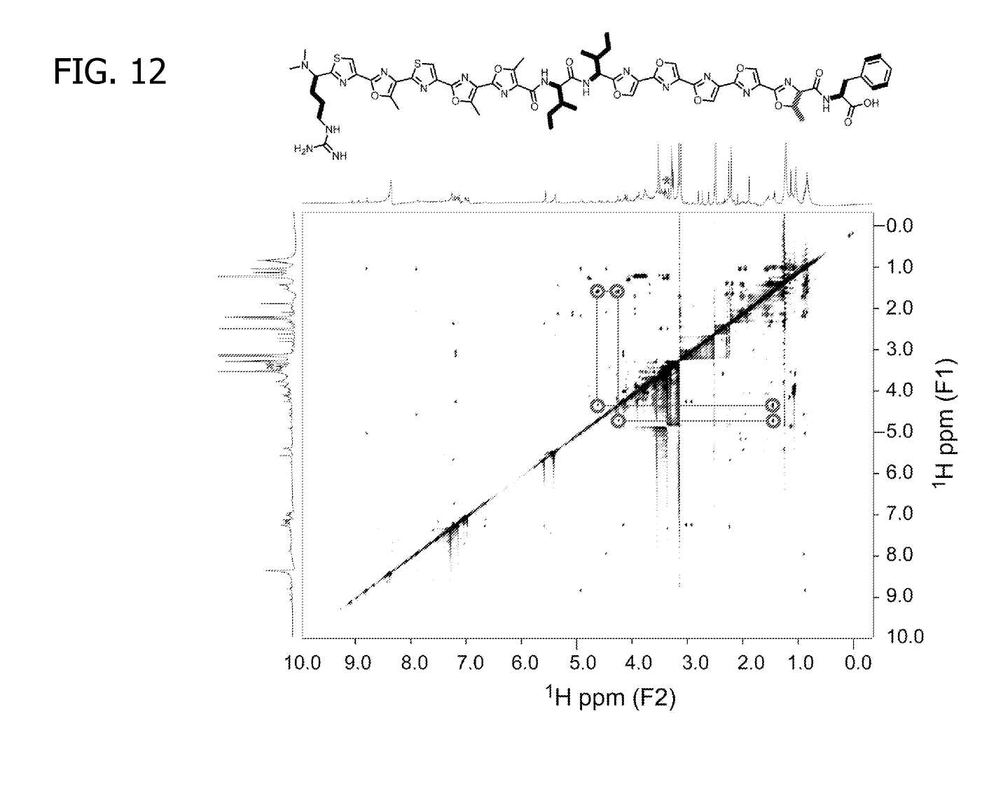

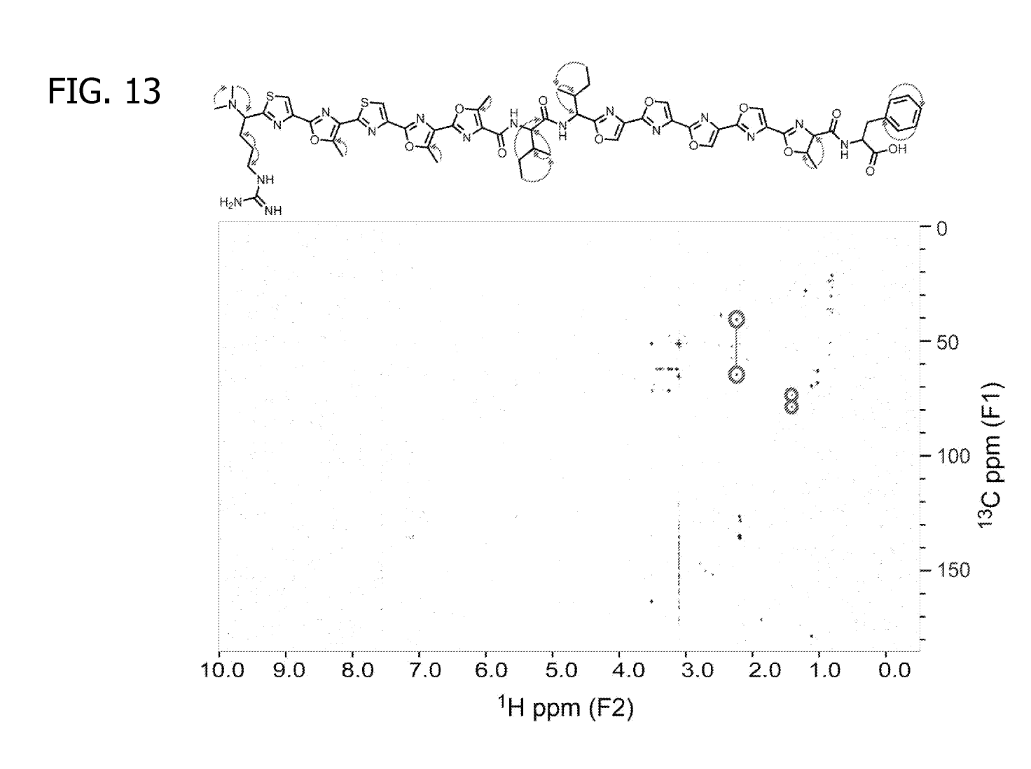

To corroborate the proposed structure elucidated by MS, a series of two-dimensional NMR experiments was performed, including ¹H-¹H-gCOSY, ¹H-¹H-TOCSY, and ¹H-¹³C-gHMBC on a 600 MHz instrument (FIGS. 11-13). Briefly, the gCOSY and TOCSY spectra confirmed the following: i. due to the absence of NH and C_{α}H correlations, all Cys, Ser, and Thr were heterocyclized (the NH and C_{α}H correlations were readily visible for all internal residues with an intact amide bond—Ile, Ile, Phe); ii. the carbon framework of the Arg1, Ile7, Ile8, and Phe14 side chains were not modified and, iii. the sole azoline moiety of PZN occurred on a Thr. The ¹H-¹³C-gHMBC spectrum further validated findings from the ¹H-¹H experiments, in addition to proving the methylation sites as N^{α},N^{α}-dimethylArg (FIG. 13). N-terminal methylation of ribosomally produced peptides in bacteria is an exceedingly rare posttranslational modification. While N-terminal dimethylation has been described on Ala (e.g. cypemycin), N^{α},N^{α}-dimethylArg appears to be a novel posttranslational modification (Garavelli, J. S. 2004, Proteomics 4, 1527-1533).

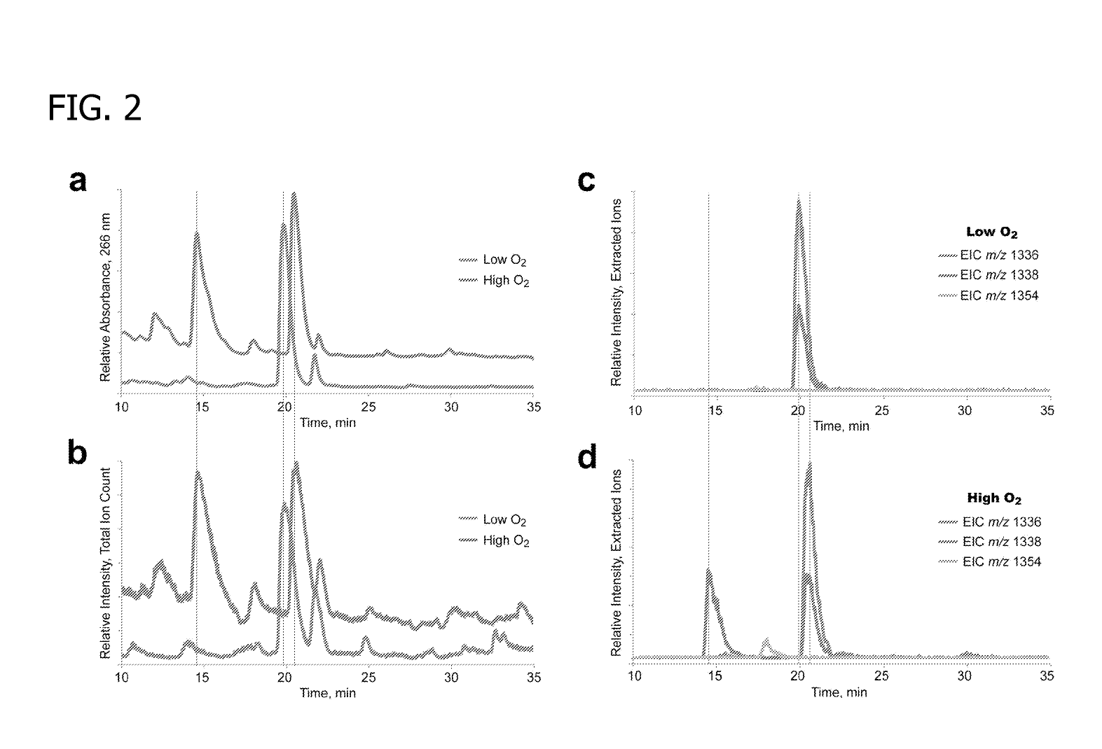

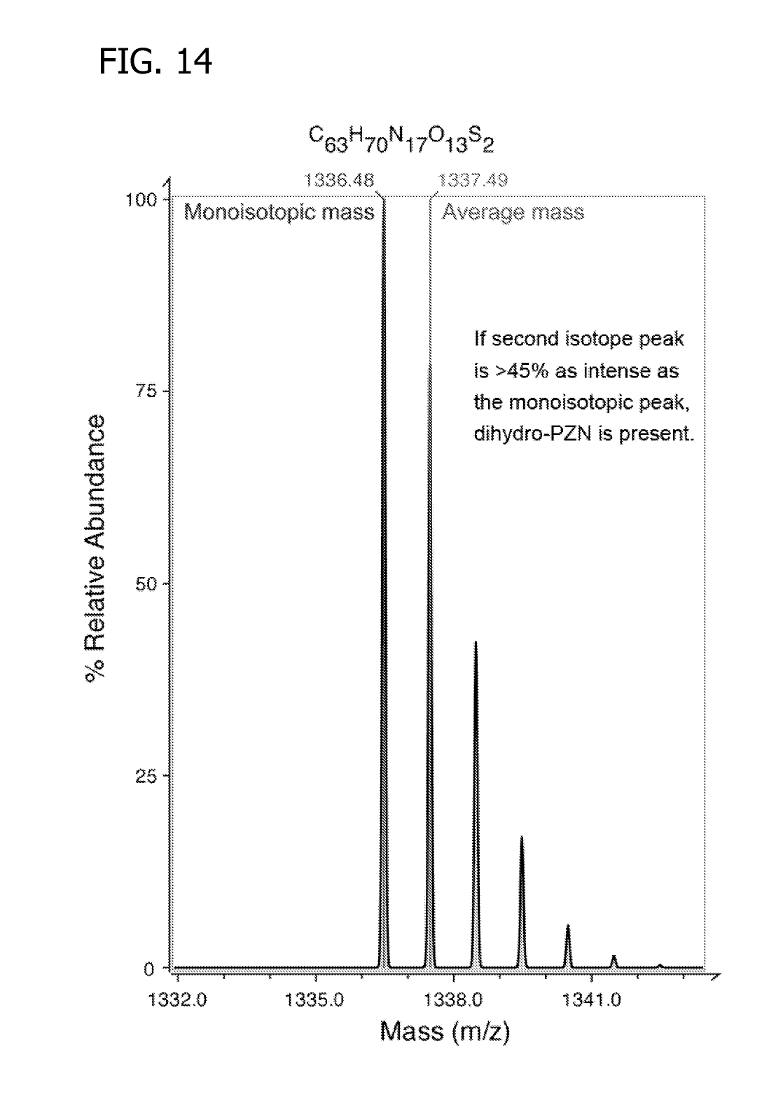

During the course of optimizing the production of PZN for detailed spectroscopic analysis, it was noticed that the level of culture oxygenation had an impact on the production of PZN and derivative metabolites. Under low oxygen fermentation, PZN (m/z 1336) was the majority species present after a non-lytic, cell surface extraction procedure, as demonstrated by the UV trace, total ion chromatogram (TIC), and the extracted ion chromatogram (EIC, FIG. 2a-c). The product of methyloxazoline ring opening (i.e. hydrolyzed PZN, m/z 1354) was also monitored (FIG. 2C-2D). The m/z 1338 species that “coeluted” with 1336 at 19.9 min was actually the second isotope peak (two extra neutrons) of m/z 1336 (FIG. 14).

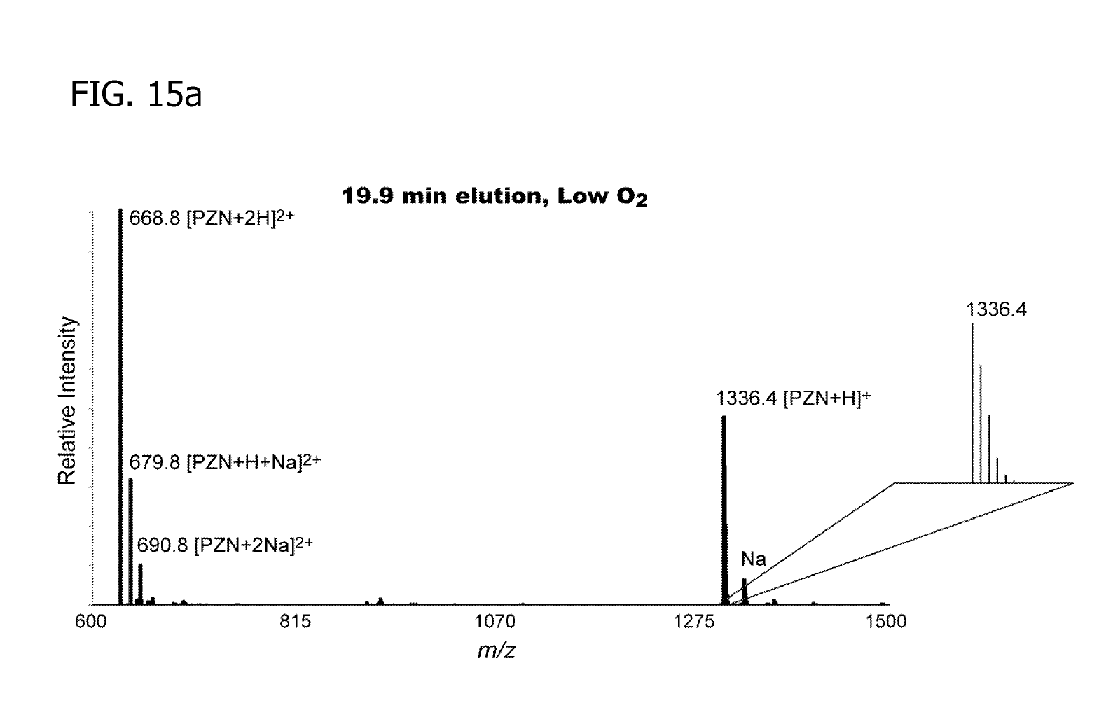

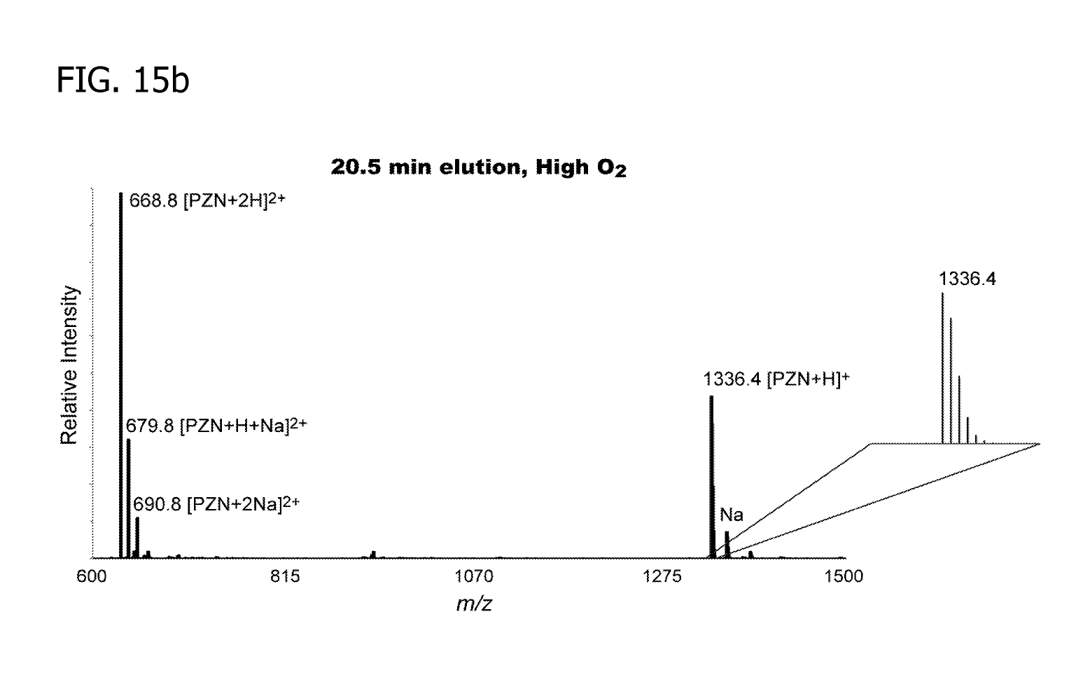

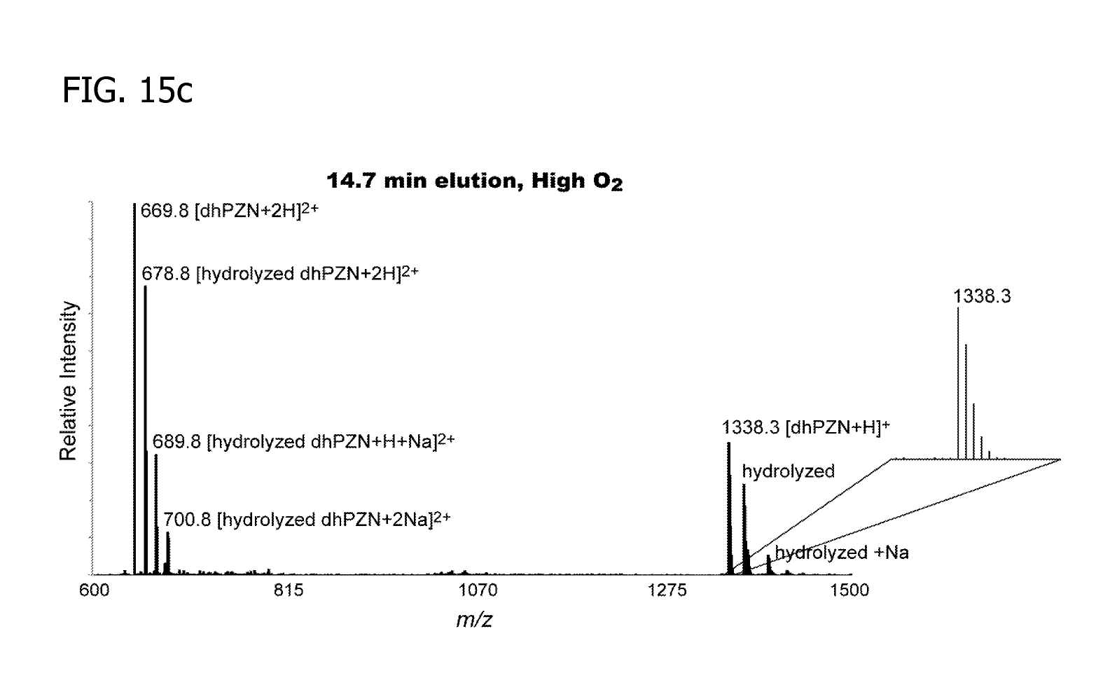

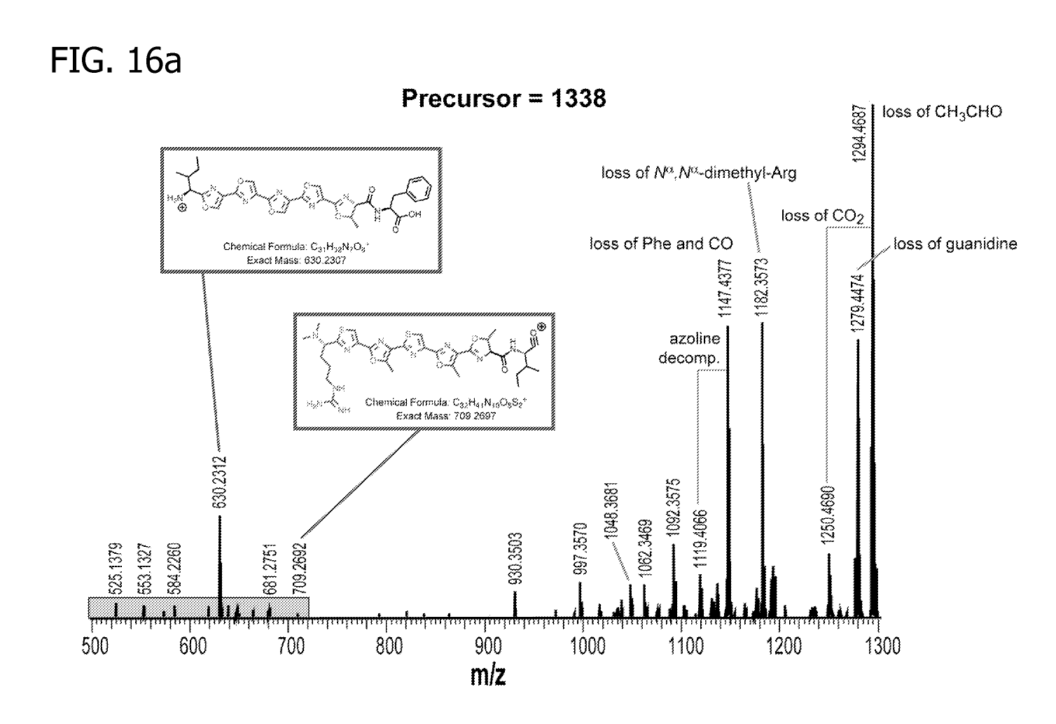

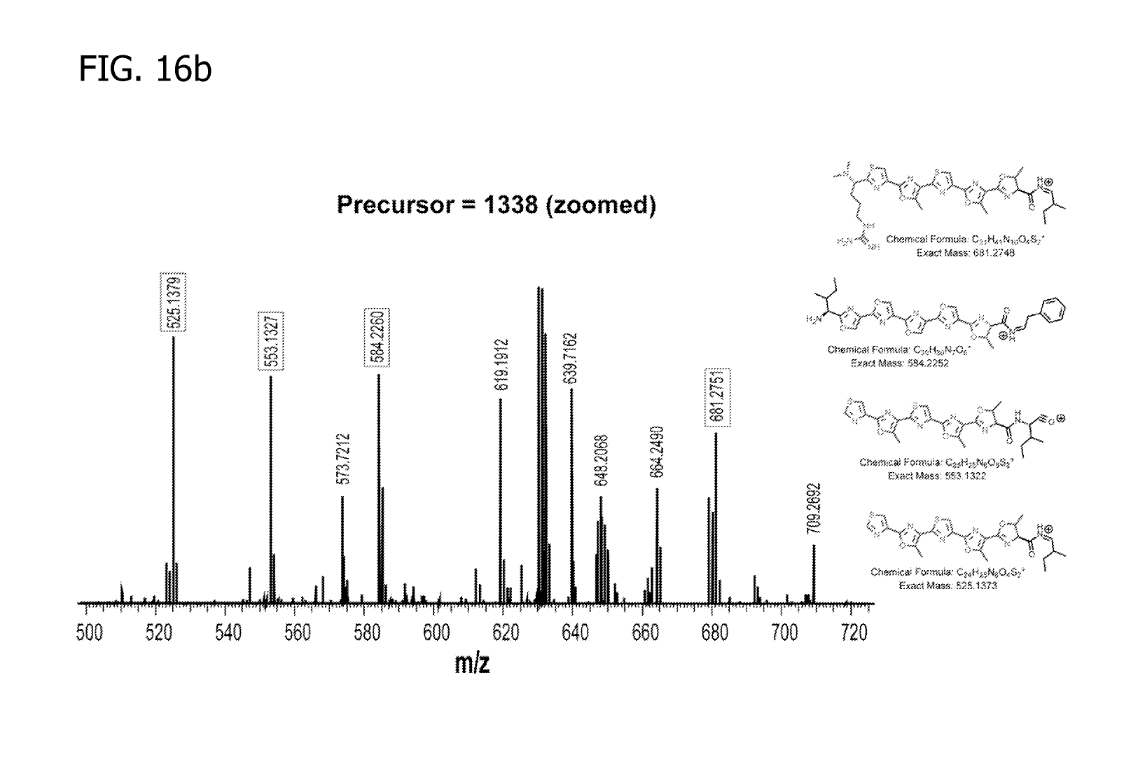

Under oxygen saturated cultivation, UV and TIC monitoring revealed an additional, highly abundant species at 14.7 min (FIG. 2A-2B). MS analysis demonstrated this species was m/z 1338, suggestive of a reduced PZN species (dihydroPZN) containing two azoline heterocycles (FIG. 2D, FIG. 15). The earlier elution time on reverse-phase chromatography suggested that this species was more polar than PZN, which was consistent with the replacement of an aromatic azole with a protonated azoline (expected in 0.1% formic acid). After treatment of m/z 1338 with mild aqueous acid, two additions of water were observed (m/z 1356 and 1374). Tandem MS was then used to demonstrate that the second azoline site was located on the N-terminal half of PZN (FIG. 16). Higher order CID analysis prompted the neutral loss of acetaldehyde, indicating that the second azoline heterocycle was derived from Thr, likely the residue directly preceding Ile (Thr6, data not shown). To an approximation, this position was sterically and electronically equivalent to the previously discussed methyloxazoline (Thr13) since both lie between an N-terminal tetra-azole and a C-terminal unmodified, hydrophobic residue (FIG. 1A).

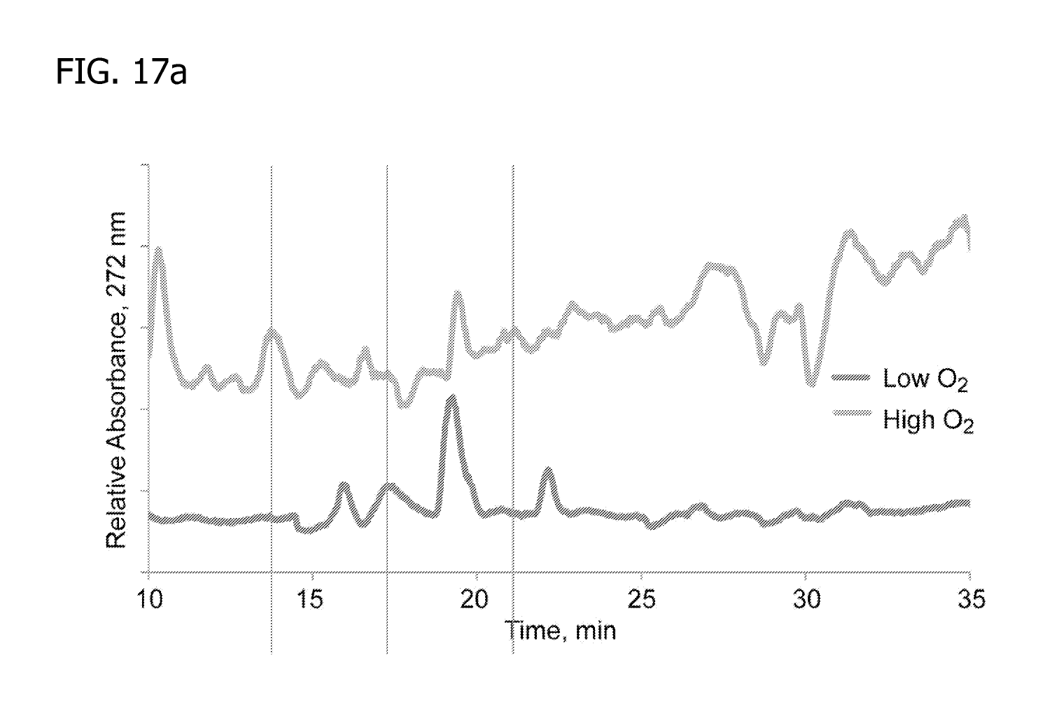

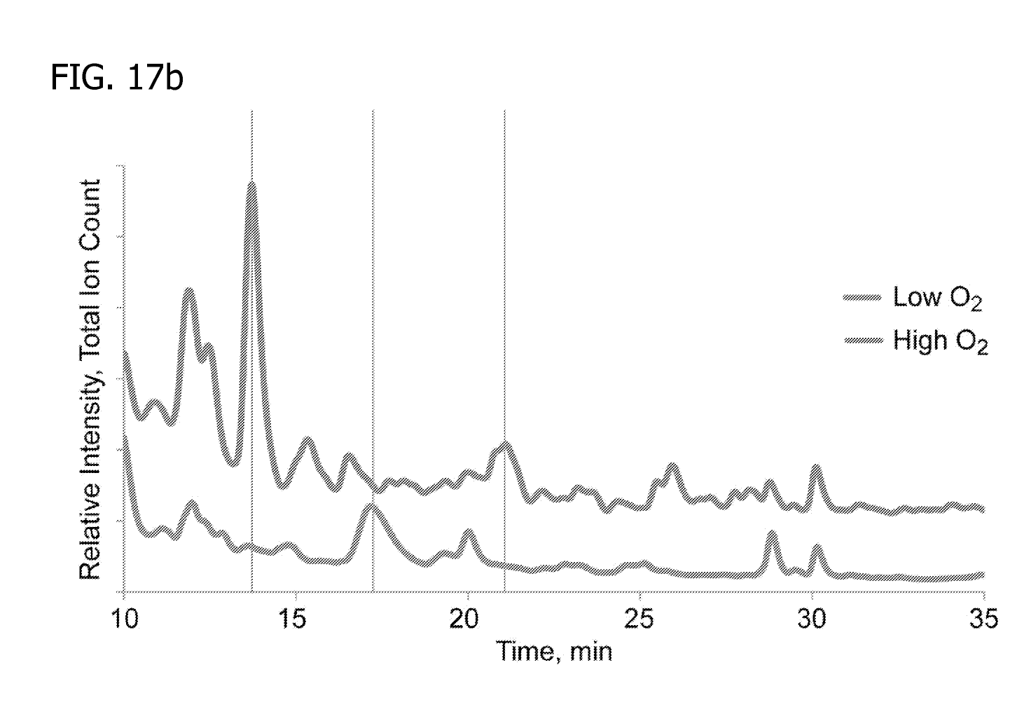

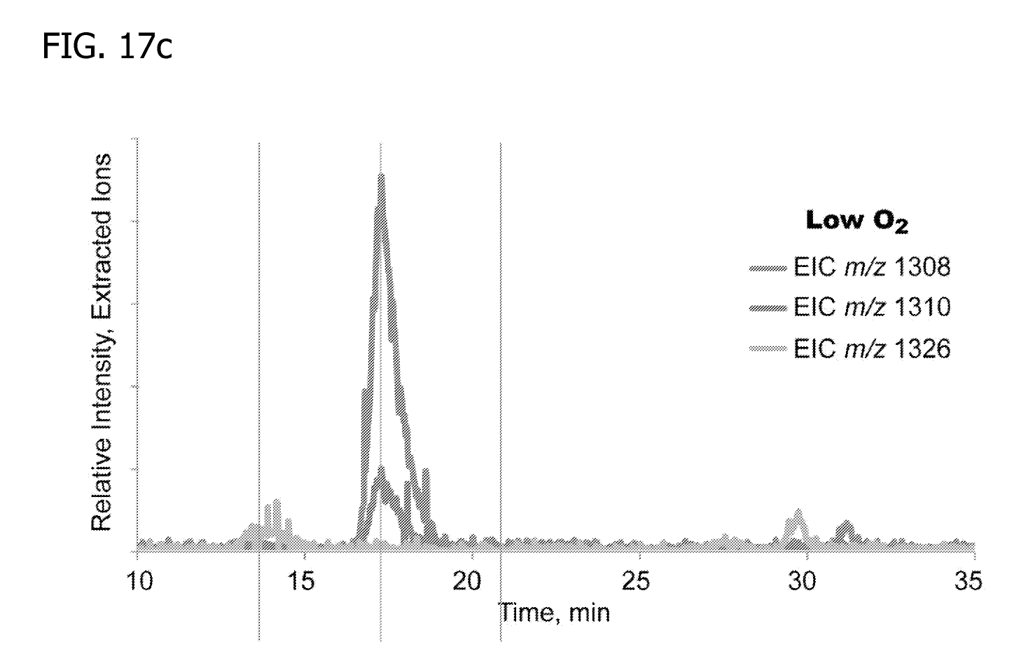

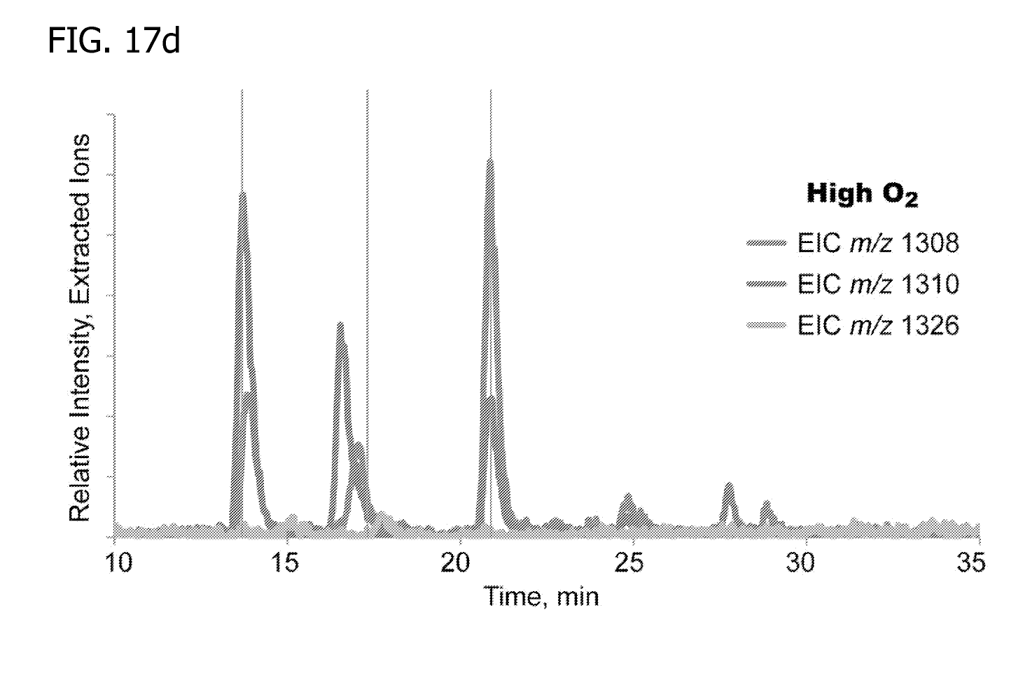

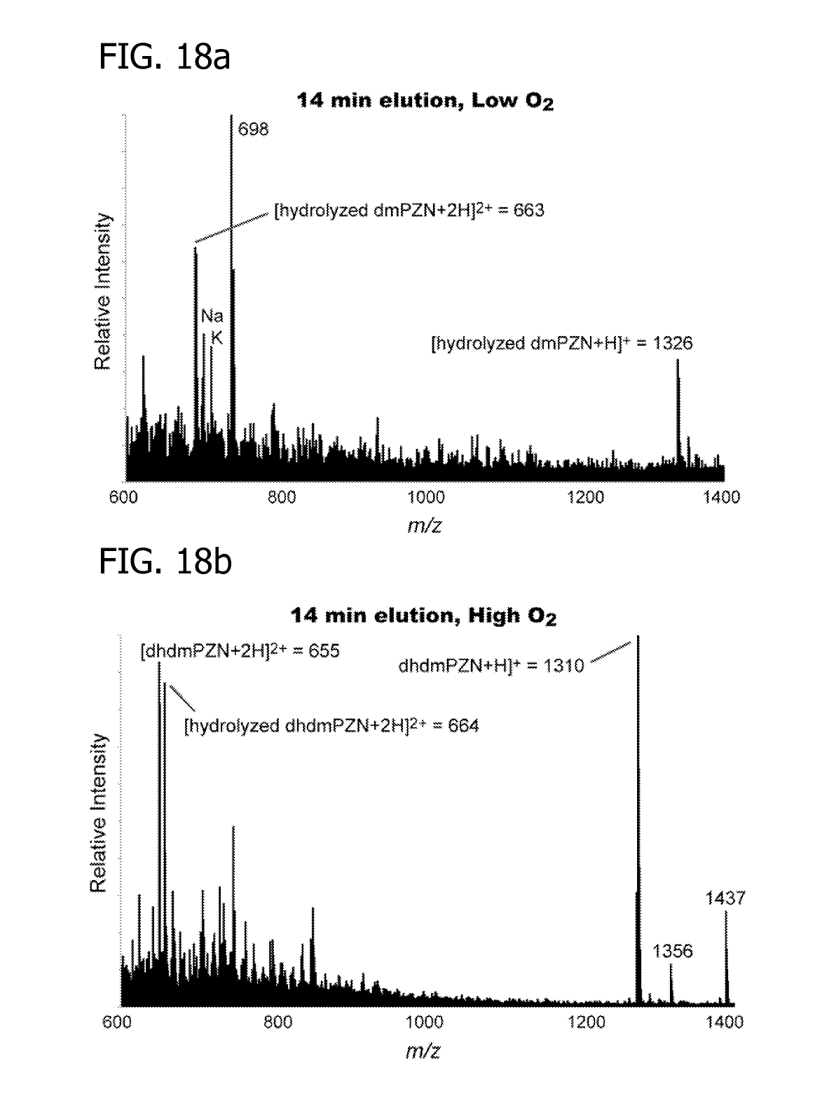

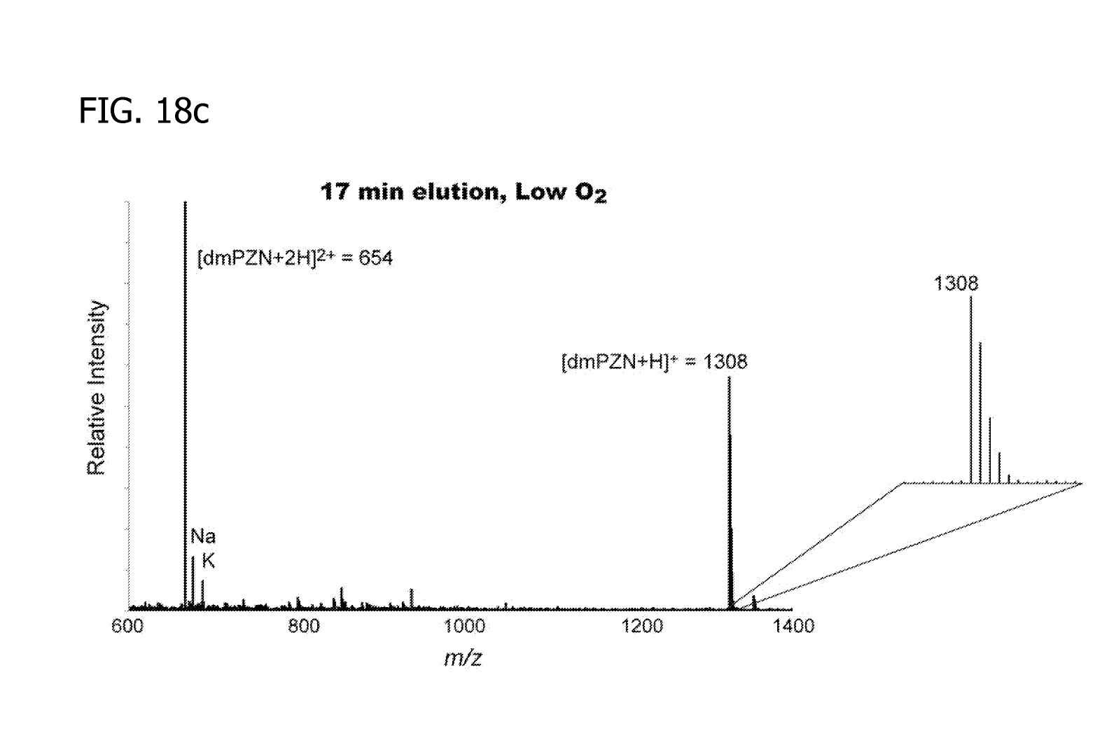

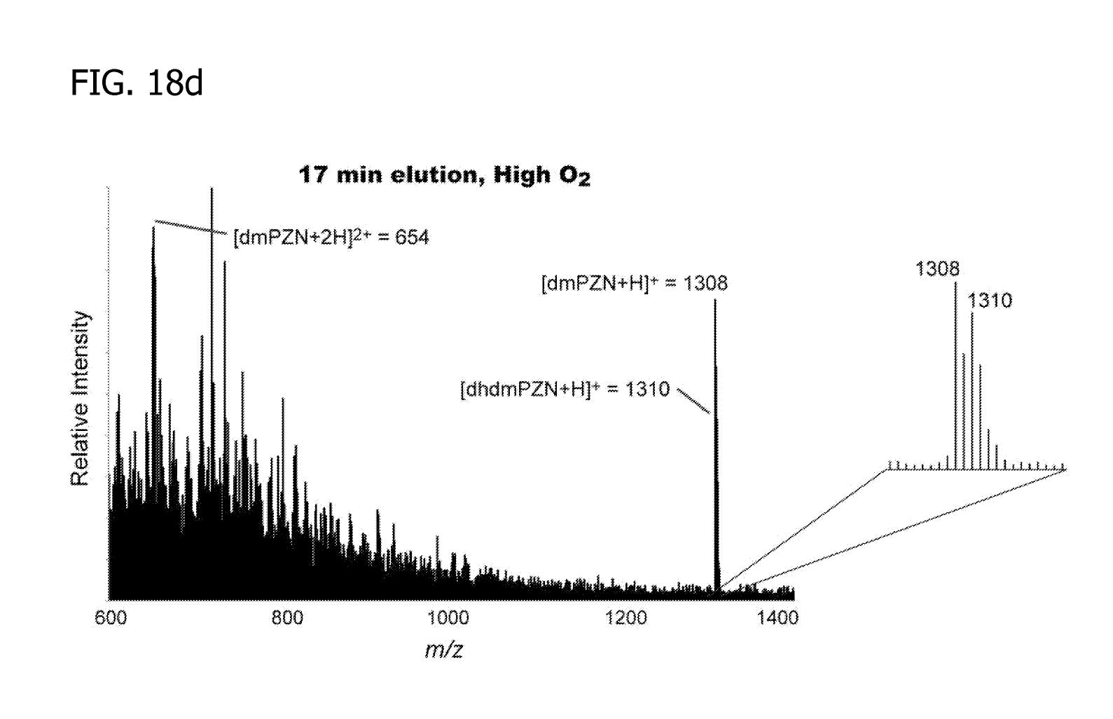

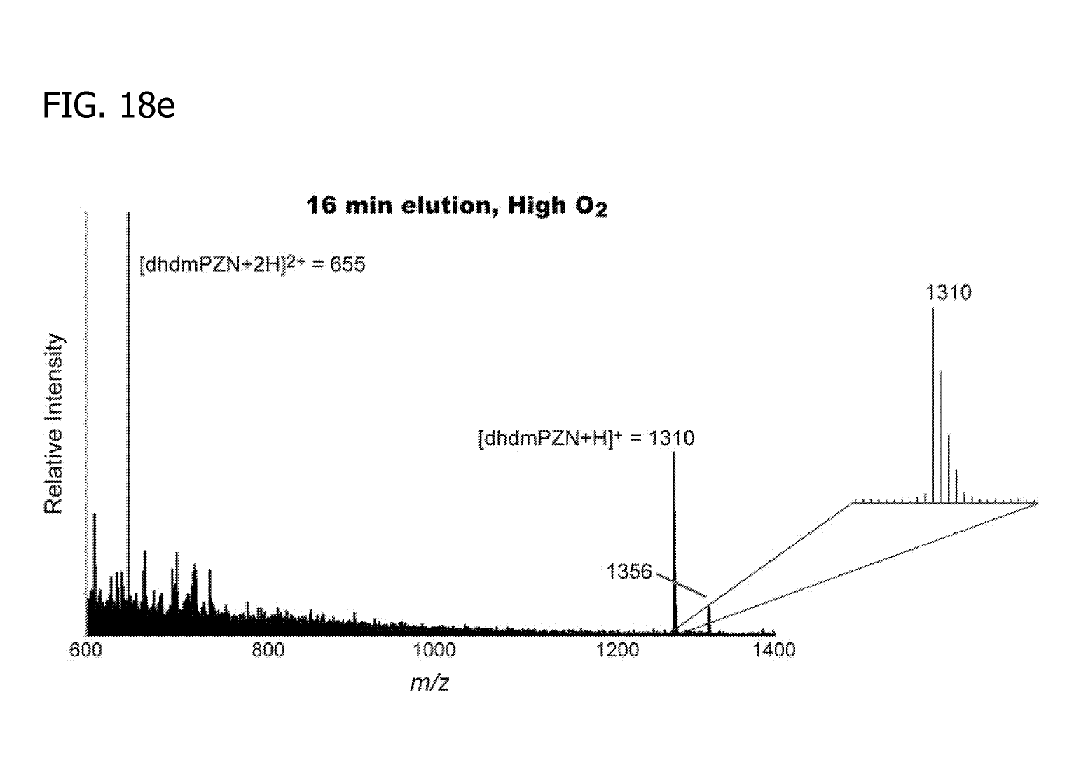

The production of additional PZN-related species was also observed for desmethylPZN when oxygenation levels were increased during cultivation of the pznL methyltransferase deletion strain (FIGS. 17-18). As above, the pznL deletion strain only produced desmethylPZN (m/z 1308) under low oxygenation. Under oxygen-saturated conditions, a significant amount of an earlier eluting species, dihydrodesmethylPZN (m/z 1310) was generated. It is possible that azoline oxidation, through the action of the FMN-containing dehydrogenase, was the rate-determining step in PZN biosynthesis. While not being bound to a particular theory, it may be possible, that with increased aeration (faster metabolism), partially processed PZN products are more rapidly produced and accepted as substrates by proteins acting downstream of the dehydrogenase (i.e. the leader peptidase, methyltransferase, and ABC transport system). The biosynthetic implication of obtaining PZN oxidation intermediates is that the rate of methyloxazoline oxidation at “Thr6” (putative) and Thr13 (FIG. 1A) is slower than the dissociation rate from the heterotrimeric synthetase (BCD) complex and subsequent maturation steps. During native PZN biosynthesis, it is most probable that “Thr6” is the last position to be oxidized. From this observed PZN oxidation intermediate, it becomes apparent that cyclodehydration precedes dehydrogenation, as has been previously supported by in vitro reconstitution experiments but never before demonstrated in vivo (McIntosh and Schmidt, 2010, Chembiochem 11, 1413-1421; Milne et al., 1999, Biochemistry 38, 4768-4781).

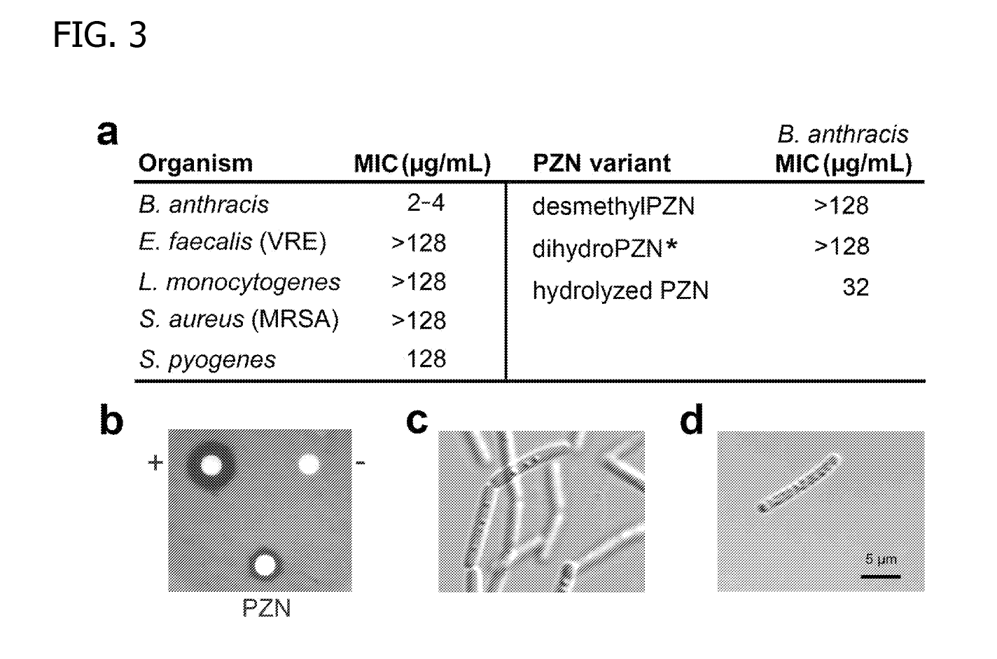

It was determined that PZN exhibited activity primarily towards Bacillus sp., including B. subtilis. PZN exhibited no activity against any tested Gram-negative organisms. To further define the selectivity within the Gram-positive organisms, the scope of PZN activity was evaluated towards a panel of ubiquitous human pathogens, including methicillin-resistant Staphylococcus aureus (MRSA), vancomycin-resistant Enterococcus faecalis (VRE), Listeria monocytogenes, Streptococcus pyogenes, and Bacillus anthracis strain Sterne (a surrogate for the BSL3 pathogen, which lacks the poly-D-glutamic acid capsule). Using a microbroth dilution bioassay, potent growth inhibition of B. anthracis was observed (FIG. 3A). All other species were unaffected by PZN, with the exception of S. pyogenes, which was only inhibited by very high concentrations of PZN.

The specificity for PZN against B. anthracis was recapitulated in an agar diffusion bioassay (FIG. 3B), as inhibition zones were not observed for any other tested bacterium (data not shown). The action of PZN upon B. anthracis was bactericidal, as reculturing of treated cells in the absence of PZN led to no bacterial growth. Live cell imaging (non-stained, non-fixed) by differential interference contrast (DIC) microscopy revealed that B. anthracis treated with PZN at 4 μg/mL underwent massive lysis, as evidenced by an abundance of cellular debris (data not shown). Of the few remaining cells that survived, marked changes were observed in the appearance of the cell surface (FIG. 3C-3D). While not being bound to a particular theory, it is believed that PZN either directly or indirectly disrupts peptidoglycan biosynthesis leading to the cell wall becoming structurally compromised.

Dimethylation of the α-amino group was apparently important for PZN's antibiotic activity, as desmethylPZN was devoid of activity against B. anthracis in both bioassays (FIG. 3A). While the molecular basis for this effect is not currently known, dimethylation increases amine basicity, increases lipophilicity, and removes two potential H-bond donors. Also tested were the effects of hydrolyzing the methyloxazoline moiety of PZN (hydrolyzed PZN, m/z 1354) and the variant with two azolines (dihydroPZN, m/z 1338). While hydrolyzed PZN retained measurable activity towards B. anthracis, dihydroPZN was devoid of activity. Due to difficulty in separating dihydroPZN from the mono- and di-hydrolyzed forms (m/z 1356 and 1374), these bioassays were performed using a 1:2:2 mixture (non:mono:di). The lack of activity with this mixture of hydrolyzed, dihydroPZN compounds might be attributable to the fact that hydrolyzed PZN is roughly 8-fold less active than PZN (FIG. 3a). It is also possible that the production of dihydroPZN is an artifact of laboratory cultivation.

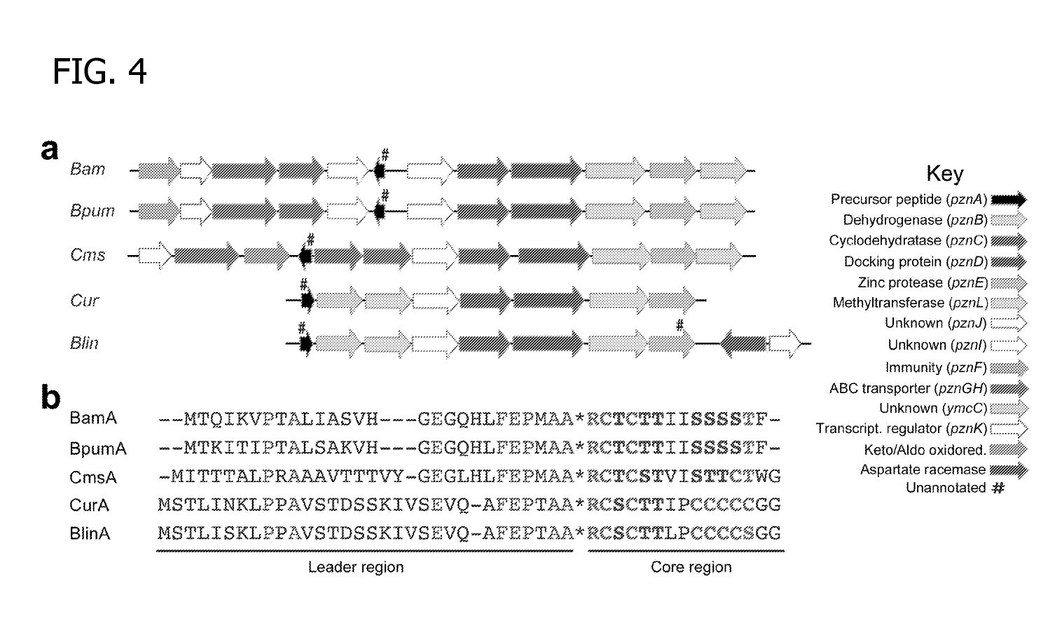

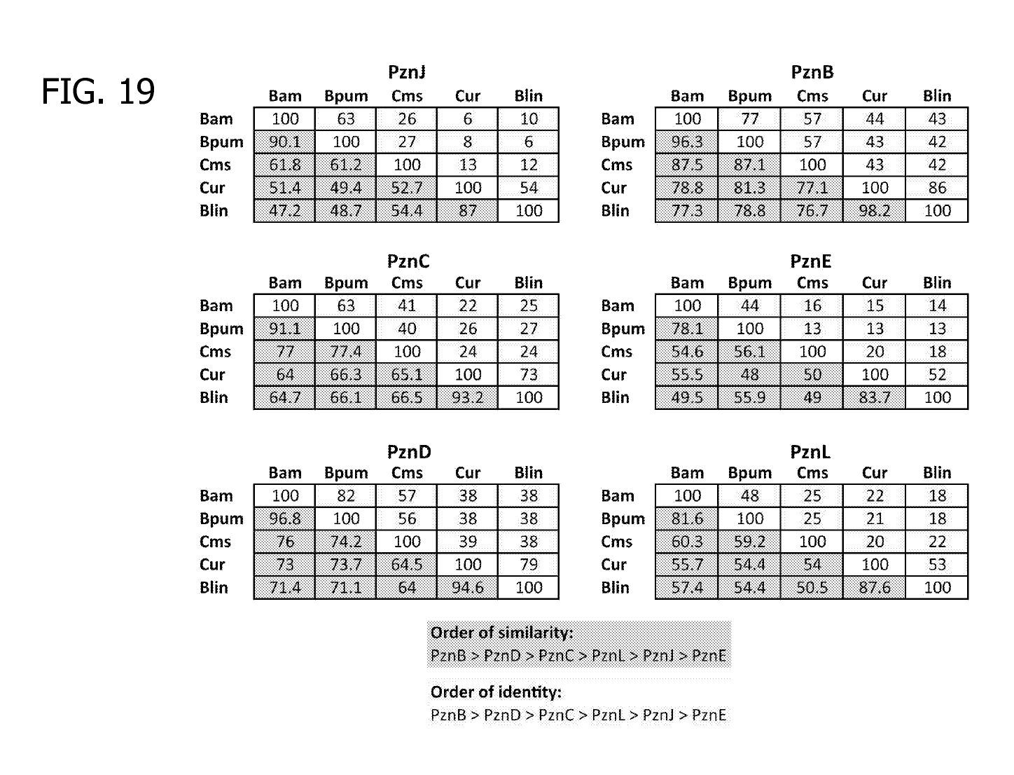

A targeted bioinformatics survey using the thiazole/oxazole synthetase proteins (cyclodehydratase, dehydrogenase, and docking protein) of PZN yielded four highly related biosynthetic gene clusters (FIG. 4). The cluster found in Bacillus pumilus ATCC 7061 (also a plant-growth promoting saprophyte) was of identical gene order and direction as the cluster from B. amyloliquefaciens FZB42. The remaining three PZN-like biosynthetic clusters were found in the Actinobacteria phylum including Clavibacter michiganensis subsp. sepedonicus (potato pathogen) (30), Corynebacterium urealyticum DSM 7109 (human skin-associated bacterium, causative agent of some urinary tract infections) and Brevibacterium linens BL2 (human skin-associated bacterium). The PZN cassettes from C. urealyticum and B. linens have amino acid similarity values much higher with each other than the other PZN producers (FIG. 19). In each of the five species, the PZN biosynthetic cluster contained the canonical TOMM genes: a precursor peptide, dehydrogenase, cyclodehydratase, and docking protein. Beyond this, all five clusters also include a putative membrane-spanning leader peptidase from the type II CAAX superfamily (31), SAM-dependent methyltransferase, and a required protein of unknown function. Conversely, homologs of the PznF immunity protein and PznGH transporters were not found in the local genomic context for the PZN biosynthetic gene clusters for C. urealyticum and B. linens (FIG. 4a). This suggests a distinct mechanism of immunity and chromosomally distant transporters for these PZN variants. Alternatively, the PZNs from C. urealyticum and B. linens could act intracellularly or the biosynthetic gene cluster might always be silent (non-product forming).

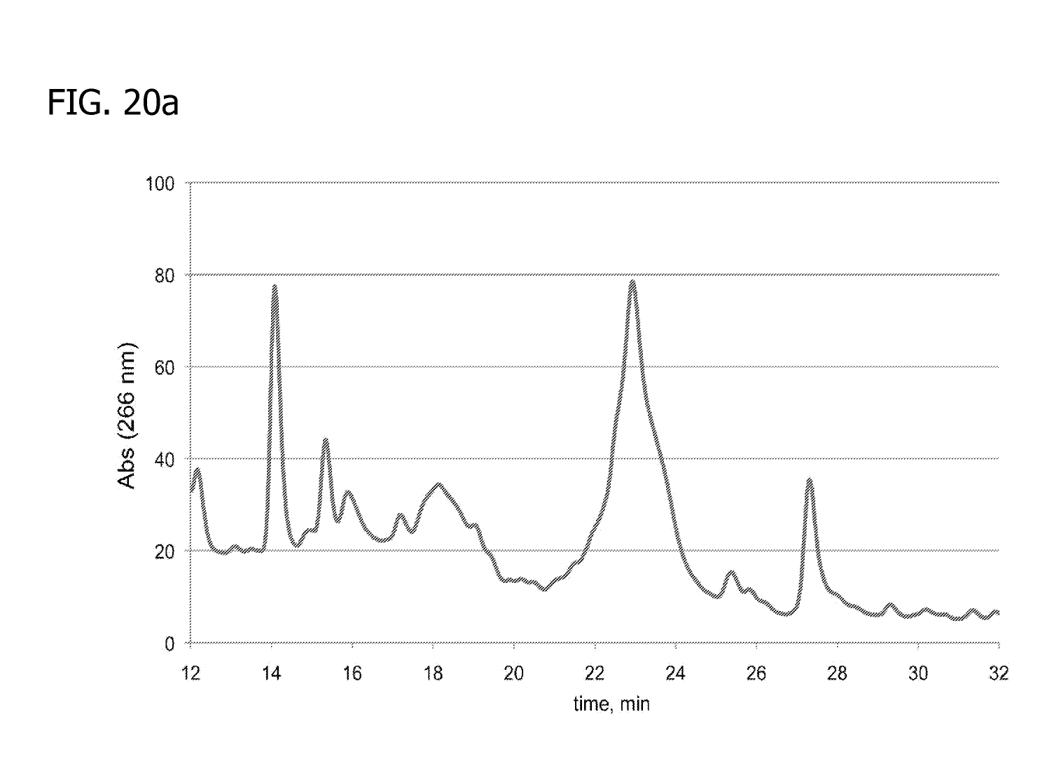

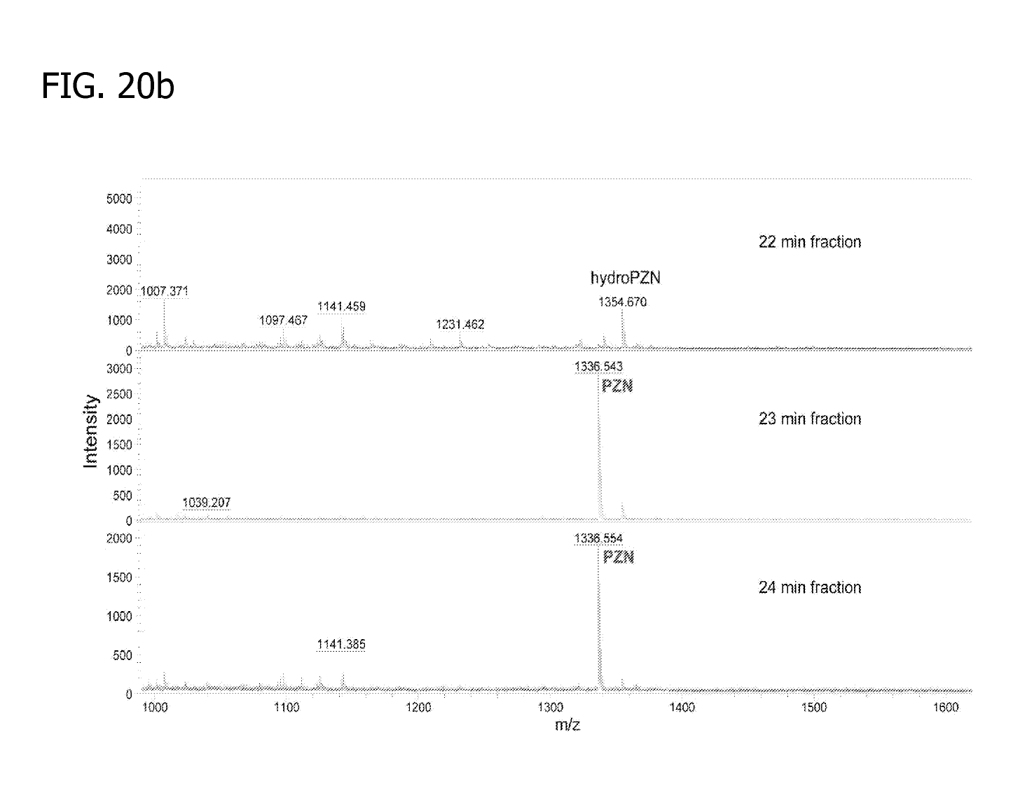

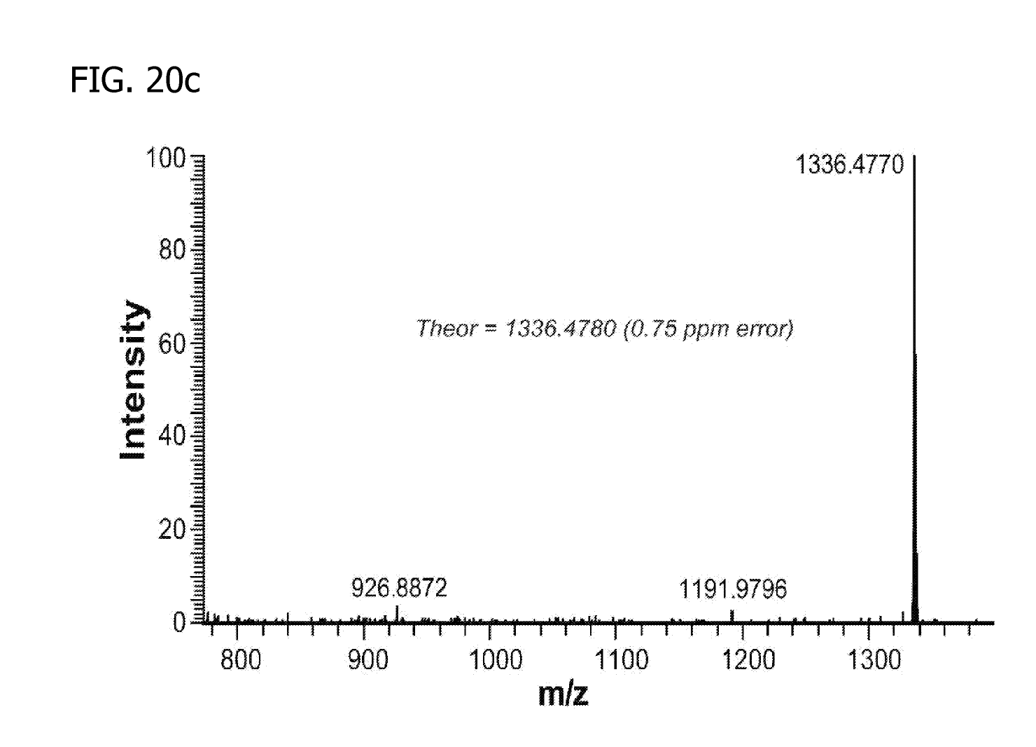

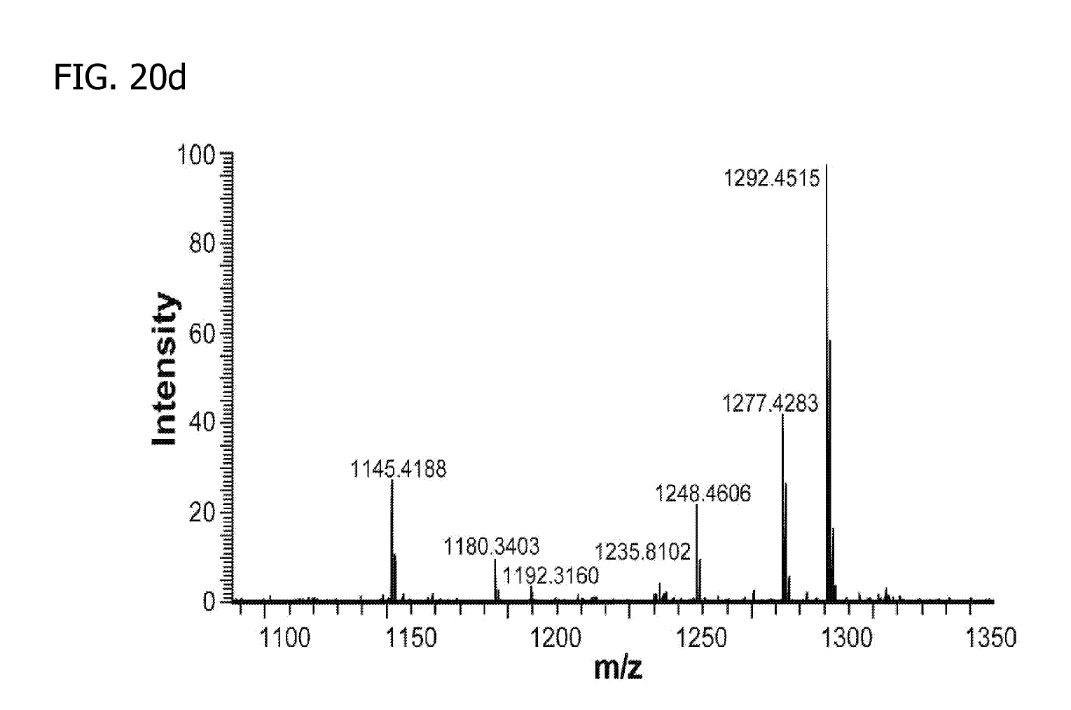

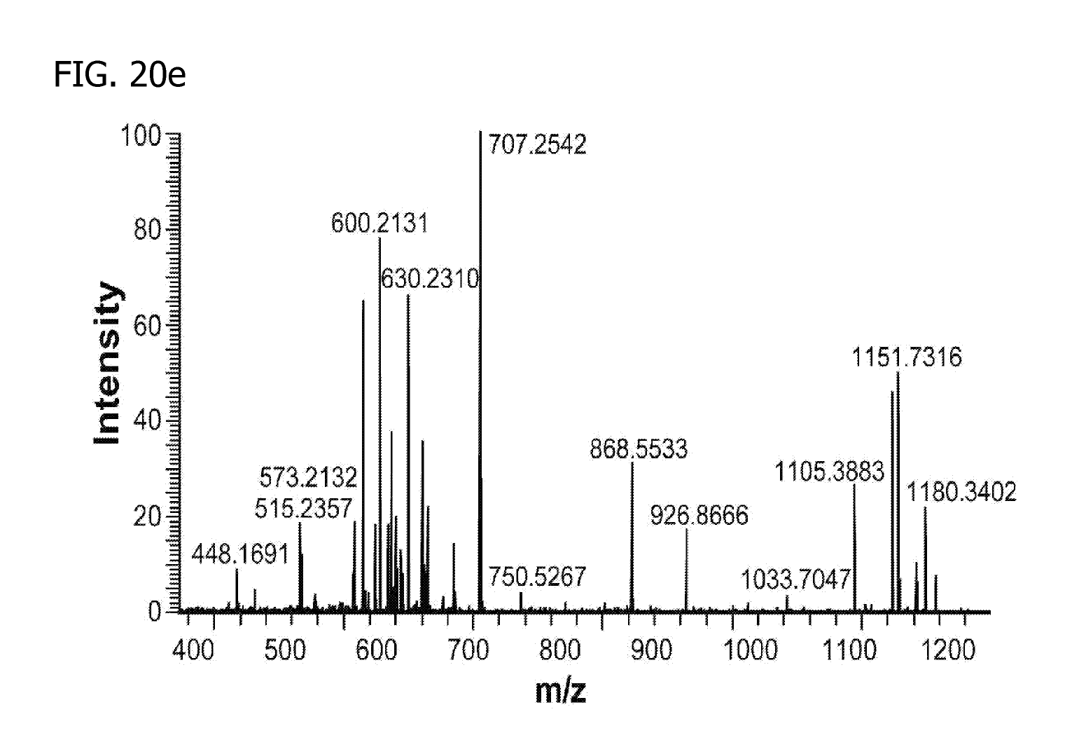

Based on the identical amino acid sequence of the core regions of the precursor peptides from FZB42 and B. pumilus, it would have been expected that these species produced identical compounds (FIG. 4B). To directly test if B. pumilus was indeed producing PZN, stationary phase B. pumilus ATCC 7061 cultures were cell surface extracted in an identical manner as with FZB42. MALDI-TOF-MS of HPLC-purified fractions revealed the presence of m/z 1336, and in an earlier fraction, m/z 1354 (+H₂O), supporting the production of PZN and hydrolyzed PZN from this strain (FIG. 20a-b). The identity of this species as PZN was confirmed by high accuracy mass measurement (LTQ-FT-MS) and CID analysis (FIG. 20c-e). As anticipated, B. amylo. FZB42 and B. pumilus ATCC 7061 were not susceptible to the action of PZN (no observable inhibition at 128 μg/mL). A non-plant associated strain of B. amylo. (NRRL B-14393), which does not produce PZN, was also completely resistant (data not shown). Resistance within the Bacillus genus to PZN is clearly complex, with a few strains being bona fide PZN producers and others simply harboring the immunity gene [e.g. B. amylo. strains YAU-Y2 and NAU-B3 and B. atrophaeus 1942, BATR1942_{—}01200, 94% identical to FZB42] (Scholz et al., supra). Early attempts to isolate a PZN-type natural product from the Actinobacteria family members were not successful. The lack of a signal by MALDI-MS and reverse transcriptase-PCR suggested that the biosynthetic genes were not transcribed under tested cultivation conditions (data not shown). As with many “silent” gene clusters, highly precise environmental conditions may be necessary for the bacterium to produce particular natural products.

Sequence alignment of all five PZN precursor peptides showed that there has been evolutionary pressure to maintain a nearly invariant chemotype giving rise to the PZN structure (from N- to C-terminus): leader peptide cleavage site and N-terminal Arg (FEPxAA*R), five cyclizable residues with position 2 and 4 always Cys and position 6 always Thr, two hydrophobic residues, five cyclizable residues, and a more variable C-terminus that ends with Phe, Trp-Gly, or Gly-Gly (FIG. 4B).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

1. A plantazolicin-like compound having the structure:
(X¹)—(X²)₅—(X³)₂—(X⁴)₅—(X⁵)ₙ
or a pharmaceutically acceptable salt or ester thereof,
wherein
X¹ is 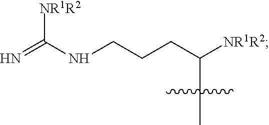
R¹ and R² are each independently hydrogen or lower alkyl;
each X² is independently an azole;
each X³ is independently a hydrophobic amino acid;
each X⁴ is independently an azole or azoline; and
each X⁵ is independently an amino acid, wherein n is 1 or 2.

2. The compound of claim 1 wherein the compound includes at least one of the following: R¹ and R² are each independently hydrogen or methyl; each X² is independently selected from the group consisting of: pyrazole, imidazole, thiazole, oxazole, isoxazole, isothiazole, pyrrole, triazole, tetrazole, and pentazole; each X³ is independently selected from the group consisting of alanine (Ala), valine (Val), isoleucine (Ile), leucine (Leu), methionine (Met), phenylalanine (Phe), tyrosine (Tyr) and tryptophan (Trp); each X⁴ is independently an azole selected from the group consisting of pyrazole, imidazole, thiazole, oxazole, isoxazole, isothiazole, pyrrole, triazole, tetrazole, and pentazole, or an azoline selected from the group consisting of pyrazoline, imidazoline, thiazoline, oxazoline, isoxazoline, isothiazoline, pyrroline, triazoline, tetrazoline, and pentazoline; each X⁵ is independently selected from the group consisting of phenylalanine, tyrosine and tryptophan; or n is 1 or 2.

3. (canceled)

4. The compound of claim 2 wherein each X² is independently thiazole or oxazole; each X³ is independently isoleucine, phenylalanine or tryptophan; each X⁴ is independently thiazole, oxazole or oxazoline; or X⁵ is phenylalanine.

5.-12. (canceled)

13. The compound of claim 1 wherein the compound is plantazolicin having the structure: 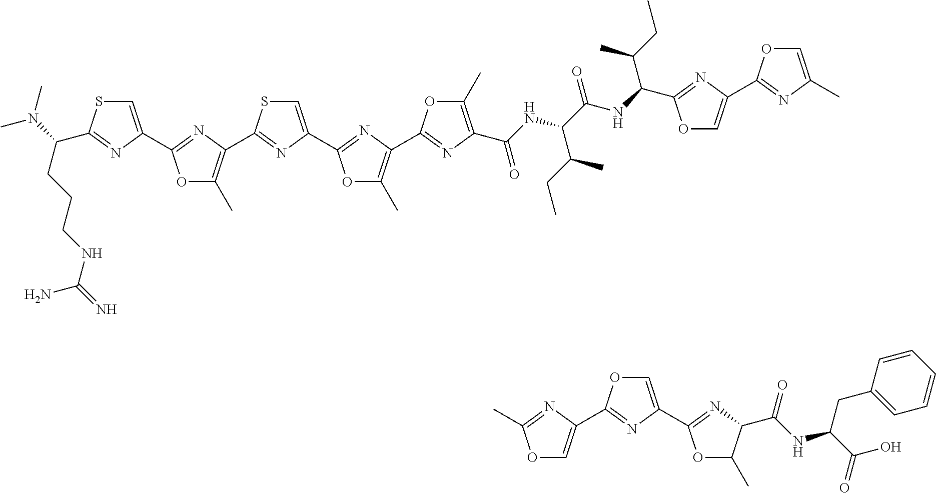
or a pharmaceutically acceptable salt or ester thereof.

14. A pharmaceutical composition comprising the compound of claim 13 and a pharmaceutically acceptable carrier.

15. The pharmaceutical composition of claim 14, wherein the composition comprises from about 100 μg to about 100 mg of the compound.

16. The pharmaceutical composition of claim 14, wherein the composition comprises from about 1 mg to about 50 mg of the compound.

17. (canceled)

18. A method for treating or preventing a Bacillus anthracis infection or a Bacillus cereus infection, the method comprising administering a pharmaceutical composition of claim 14 to an animal subject in need thereof.

19. The method of claim 18, wherein the animal subject is infected with anthrax spores and has anthrax disease.

20. The method of claim 18, wherein the animal subject is a human.

21. The method of claim 18, wherein the animal subject is a dog, cat, horse, sheep, goat, or cow.

22. The method of claim 18, wherein the anthrax is inhalation anthrax, cutaneous anthrax, oropharyngeal anthrax or gastrointestinal anthrax.

23. The method of claim 18, wherein the composition is administered orally, intravenously, intramuscularly or subcutaneously.

24.-25. (canceled)

26. The method of claim 18, wherein the composition is administered once, twice or three times a day.

27.-32. (canceled)

33. A method for identifying a plantazolicin-like protein, wherein the method comprises:
identifying a bacterial amino acid sequence exhibiting at least 50% amino acid identity to a plantazolicin precursor peptide from Bacillus amyloliquefaciens FZB42;
obtaining a post-translationally modified product of the bacterial amino acid sequence; and
testing the post-translationally modified product of the bacterial amino acid sequence in a Bacillus anthracis growth inhibitory assay, wherein ability to inhibit the growth of Bacillus anthracis indicates that the bacterial amino acid sequence encodes a plantazolicin-like protein.

34.-35. (canceled)

36. A method for producing the plantazolicin compound of claim 13, the method comprising:
growing Bacillus cells in culture;
collecting the Bacillus cells, thereby obtaining the harvested Bacillus cells;
obtaining a crude plantazolicin extract from the harvested Bacillus cells; and
purifying the plantazolicin compound from the crude plantazolicin extract, wherein the Bacillus cells are Bacillus amyloliquefaciens FZB42 cells or Bacillus pumilus cells.

37. (canceled)

38. The method of claim 36, wherein growing cells in culture is performed under low oxygenation conditions; collecting the cells is performed by centrifugation; obtaining the crude plantazolicin extract is performed by methanolic extraction; or purifying the plantazolicin compound is performed by high performance liquid chromatography (HPLC).

39.-42. (canceled)

43. The method of claim 36, further comprising the step of converting the purified plantazolicin compound to a salt or an ester thereof.

44. A method of identifying Bacillus anthracis comprising:
combining the compound of claim 1 with a biological sample suspected of containing a human pathogen to form a mixture; and
analyzing the mixture to determine the extent of cell lysis or cell growth inhibition, wherein growth inhibition or lysis of a majority of cells in the sample is indicative of the presence of Bacillus anthracis.

45. The method of claim 44 wherein the compound is the plantazolicin compound of claim 13.

46. The method of claim 44 wherein the extent of cell lysis is analyzed wherein lysis of a majority of cells in the sample is indicative of the presence of Bacillus anthracis.

47. The method of claim 46 wherein the mixture is analyzed via live cell imaging.

48. The method of claim 47 wherein the live cell imaging is differential interference contrast (DIC) microscopy.

49. The method of claim 48 wherein the mixture is analyzed to determine the extent of cell growth inhibition, wherein growth inhibition is indicative of the presence of Bacillus anthracis.

50. The method of claim 49 wherein the extent of growth inhibition of the cultured mixture is analyzed via a microbroth dilution assay.

51. The method of claim 49 further comprising culturing the mixture prior to analyzing the extent of growth inhibition of the cultured mixture via an agar disk diffusion assay.

52. The method of claim 49 wherein the extent of growth inhibition of the mixture is analyzed by visual observation of a clear zone in the mixture if growth inhibition has occurred.


SEQUENCE LISTING (58 sequences)

SEQ ID NO: 1 - protein, 41 aa, Bacillus amyloliquefaciens
MTQIKVPTALIASVHGEGQHLFEPMAARCTCTTIISSSSTF

SEQ ID NO: 2 - protein, 14 aa, Bacillus amyloliquefaciens
RCTCTTIISSSSTF

SEQ ID NO: 3 - DNA, 22 bp, Artificial Sequence
  Spc-fw primer
ctcagtggaacgaaaactcacg

SEQ ID NO: 4 - DNA, 22 bp, Artificial Sequence
  Spc-rv
taaggtggatacacatcttgtc

SEQ ID NO: 5 - DNA, 22 bp, Artificial Sequence
  pznB-fw primer
atccatatcgccaatcatacgg

SEQ ID NO: 6 - DNA, 22 bp, Artificial Sequence
  pznB-rv
ggaatcaatacctgtcagttcg

SEQ ID NO: 7 - DNA, 22 bp, Artificial Sequence
  pznD-fw
attgactaggaggtattggacg

SEQ ID NO: 8 - DNA, 22 bp, Artificial Sequence
  pznD-rv
ttctattgaataggaggaggcg

SEQ ID NO: 9 - DNA, 22 bp, Artificial Sequence
  007400cst-fw primer
tggaatgctctttccgcagtac

SEQ ID NO: 10 - DNA, 22 bp, Artificial Sequence
  007400cst-rv primer
gtaactctgtttccacgtaacc

SEQ ID NO: 11 - DNA, 22 bp, Artificial Sequence
  7400 rv primer
tcttcatcacgcaaatcagtgc

SEQ ID NO: 12 - DNA, 22 bp, Artificial Sequence
  7400 fw primer
ccgcataaacgggaattggaag

SEQ ID NO: 13 - DNA, 48 bp, Artificial Sequence
  Spc in 7410
tctatagaaacttctctcaattagaaaagaaaagggcaaggaaatgag

SEQ ID NO: 14 - DNA, 48 bp, Artificial Sequence
  7410 in spc
actcatttccttgcccttttcttttctaattgagagaagtttctatag

SEQ ID NO: 15 - DNA, 45 bp, Artificial Sequence
  Start in spc
ctttgtaaaaagaggagcctgtcttatgagcaatttgattaacgg

SEQ ID NO: 16 - DNA, 50 bp, Artificial Sequence
  Spc in start
tttttccgttaatcaaattgctcataagacaggctcctctttttacaaag

SEQ ID NO: 17 - DNA, 45 bp, Artificial Sequence
  7430 in spc
gctgggactaaaaggagagcgggaaatgagcaatttgattaacgg

SEQ ID NO: 18 - DNA, 49 bp, Artificial Sequence
  Spc in ORF2
ttctatagaaacttctctcaattagatttaatataaagaagcatagacc

SEQ ID NO: 19 - DNA, 50 bp, Artificial Sequence
  Spc in 7430
tttttccgttaatcaaattgctcatttcccgctctccttttagtcccagc

SEQ ID NO: 20 - DNA, 48 bp, Artificial Sequence
  ORF2 in spc
tggtctatgcttctttatattaaatctaattgagagaagtttctatag

SEQ ID NO: 21 - DNA, 22 bp, Artificial Sequence
  7440 rv
tcacgtccaatacctcctagtc

SEQ ID NO: 22 - DNA, 22 bp, Artificial Sequence
  7440 fw
atcgacagagggcagattatcg

SEQ ID NO: 23 - DNA, 45 bp, Artificial Sequence
  ORF2 in spc for 7450 fw
gattattgactaggaggtattggacatgagcaatttgattaacgg

SEQ ID NO: 24 - DNA, 49 bp, Artificial Sequence
  7460 in spc for 7450 rv
gtttgttgagacatctgtattcctccctaattgagagaagtttctatag

SEQ ID NO: 25 - DNA, 22 bp, Artificial Sequence
  7450 rv
taatgtcgtccatttactcacc

SEQ ID NO: 26 - DNA, 22 bp, Artificial Sequence
  7450 fw
ttggctcgaataaatgttgacc

SEQ ID NO: 27 - DNA, 50 bp, Artificial Sequence
  Spc in ORF2 for 7450 rv
tttttccgttaatcaaattgctcatgtccaatacctcctagtcaataatc

SEQ ID NO: 28 - DNA, 51 bp, Artificial Sequence
  Spc in 7460 for 7450 fw
ttctatagaaacttctctcaattagggaggaatacagatgtctcaacaaac

SEQ ID NO: 29 - DNA, 50 bp, Artificial Sequence
  End in spc for 7500 rv
cgcttagaccctaaagatatactttctctaattgagagaagtttctatag

SEQ ID NO: 30 - DNA, 45 bp, Artificial Sequence
  7490 in spc for 7500 fw
aactctttggaggtgtcacagttatatgagcaatttgattaacgg

SEQ ID NO: 31 - DNA, 22 bp, Artificial Sequence
  7500 fw
aaggtcctagacgccctattcc

SEQ ID NO: 32 - DNA, 22 bp, Artificial Sequence
  7500 rv
gatgtgtagttttcaacgctcg

SEQ ID NO: 33 - DNA, 50 bp, Artificial Sequence
  Spc in end for 7500 fw
ctatagaaacttctctcaattagagaaagtatatctttagggtctaagcg

SEQ ID NO: 34 - DNA, 49 bp, Artificial Sequence
  Spc in 7490 for 7500 rv
ccgttaatcaaattgctcatataactgtgacacctccaaagagtttacc

SEQ ID NO: 35 - DNA, 22 bp, Artificial Sequence
  pznF fw
ggattattgcgtactccgtttc

SEQ ID NO: 36 - DNA, 19 bp, Artificial Sequence
  pznF rv
ctgcctccgccaataaatg

SEQ ID NO: 37 - DNA, 19 bp, Artificial Sequence
  pznK fw
atgccaaagtacggttggg

SEQ ID NO: 38 - DNA, 20 bp, Artificial Sequence
  pznK rv
ctccttgtaggctgctttcc

SEQ ID NO: 39 - DNA, 22 bp, Artificial Sequence
  pznG fw
ccacaggatatcagccttgaag

SEQ ID NO: 40 - DNA, 20 bp, Artificial Sequence
  pznG rv
cgataatctgccctctgtcg

SEQ ID NO: 41 - DNA, 20 bp, Artificial Sequence
  pznH fw
cgctcgctcaaattgaaacg

SEQ ID NO: 42 - DNA, 20 bp, Artificial Sequence
  pznH rv
acaacaacccacagatacgc

SEQ ID NO: 43 - DNA, 20 bp, Artificial Sequence
  pznI fw
tagcctggaagcagagggta

SEQ ID NO: 44 - DNA, 20 bp, Artificial Sequence
  pznI rv
acttttggcaggtgacaacc

SEQ ID NO: 45 - DNA, 25 bp, Artificial Sequence
  pznA fw
ggaggaggtaacaattatgactcaa

SEQ ID NO: 46 - DNA, 20 bp, Artificial Sequence
  pznA rv
ggtacaggtacagcgtgcag

SEQ ID NO: 47 - DNA, 21 bp, Artificial Sequence
  pznJ fw
ttggatatcggaatcgagttg

SEQ ID NO: 48 - DNA, 20 bp, Artificial Sequence
  pznJ rv
cggatgcccaattatctgtt

SEQ ID NO: 49 - DNA, 20 bp, Artificial Sequence
  pznC fw
tcatgtcccttggttgtgtg

SEQ ID NO: 50 - DNA, 21 bp, Artificial Sequence
  pznC rv
gccgtgataccatacttgagg

SEQ ID NO: 51 - DNA, 20 bp, Artificial Sequence
  pznD fw
cgcgatgtagatgacgtttg

SEQ ID NO: 52 - DNA, 22 bp, Artificial Sequence
  pznD rv
gattggcgatatggattagttg

SEQ ID NO: 53 - DNA, 20 bp, Artificial Sequence
  pznB fw
aaggcatgccactaatttgg

SEQ ID NO: 54 - DNA, 20 bp, Artificial Sequence
  pznB rv
gataaagagctccgccagaa

SEQ ID NO: 55 - DNA, 22 bp, Artificial Sequence
  pznE fw
catagcaataatgcgtacggtg

SEQ ID NO: 56 - DNA, 19 bp, Artificial Sequence
  pznE rv
gagacattgtcggcgaaga

SEQ ID NO: 57 - DNA, 21 bp, Artificial Sequence
  pznL fw
gatgagagggaaacctcatcc

SEQ ID NO: 58 - DNA, 20 bp, Artificial Sequence
  pznL rv
ctcccaaactgttcctgtcc